(19) United States
(12) Patent Application Publication
(10) Pub. No.: US 2026/0130758 A1
(43) Pub. Date: May 14, 2026

(54) VALVE PROSTHESIS AND TRANSCATHETER DELIVERY SYSTEM

(71) Applicant: Open Stent Solution SAS, Dury (FR)

(72) Inventors: Alexandre HADIR, Dury (FR); Maurice BOULOT, Dury (FR); Fabien LOMBARDI, Dury (FR); Stephane PARSAIX, Dury (FR)

(73) Assignee: Open Stent Solution SAS, Dury (FR)

(21) Appl. No.: 19/271,031

(22) Filed: Jul. 16, 2025

Related U.S. Application Data
(63) Continuation of application No. PCT/IB2024/000039, filed on Jan. 17, 2024.
(60) Provisional application No. 63/480,616, filed on Jan. 19, 2023.

(51) Int. Cl. A61F 2/24

(52) U.S. Cl. CPC A61F 2/2418; A61F 2/2427; A61F 2220/0033; A61F 2230/0006; A61F 2230/0034; A61F 2230/0065; A61F 2230/0069; A61F 2230/0091; A61F 2250/0037; A61F 2250/0069

(57) ABSTRACT

Provided herein are devices and systems for treatment of Heart Valve Disease. In some cases, the devices can comprise valve prosthesis comprising a valve structure and an intraluminal support structure. The valve structure can comprise support bodies, central leaflet cusps, and connecting skirts. The intraluminal support structure can comprise a two-dimensional scaffold panel comprising a support beam and a circumferential rail. The support beam can be adjustable. The circumferential rail can be adjustable. The scaffold panel can comprise guiding slots and hook that slidably couple to each other, thereby forming a three-dimensional ring. The intraluminal support structure can comprise gap filling arms extending from the support beam to fill in gap between the intraluminal support structure and the native tissue.

### CROSS-REFERENCE

This application is a continuation of PCT Application No. PCT/IB2024/000039, filed Jan. 17, 2024, which claims the benefit of U.S. Provisional Application No. 63/480,616, filed Jan. 19, 2023, which application is incorporated herein by reference.

### BACKGROUND

Heart Valve Disease (HVD) affects many people globally. HVD can manifest in abnormal valve leaflet tissue in various ways, including excess tissue growth, tissue degradation, tissue rupture, tissue hardening, tissue calcification, abnormal tissue re-positioning in response to cardiac configuration during different stages of the cardiac cycle, for example annular dilation or ventricular reshaping. Such abnormal tissue often leads to degradation of valve function, for example, leakage, backflow as a result of valve insufficiency, resistance to blood forward flow as a result of valve stenosis, and the like.

### SUMMARY

Provided herein are devices and systems for treating Heart Valve Disease.

Disclosed herein is an intraluminal support structure for deployment into a heart valve of a subject, comprising a scaffold panel having a first free end and a second free end when the scaffold panel is in a flattened configuration. The first and second free ends can be configured to be coupled to each other to form into a ring when deployed. The scaffold panel can comprise one or more support beams traversing the scaffold pattern between the first free end to the second free end at an inflow side of the scaffold panel. The scaffold panel can comprise one or more circumferential rails traversing the scaffold pattern between the first free end to the second free end at an outflow side of the scaffold panel. In some cases, a length of the one or more support beams, a length of the one or more circumferential rails, or both is adjustable.

The support structure can comprise one or more adjustable support beams and one or more non-adjustable circumferential rails. The perimeter of the one or more adjustable support beams can be less than a perimeter of the heart valve. The support structure when deployed into the heart valve can generate a chronic force proportional to the difference between a perimeter of the support structure and the perimeter of the heart valve. The support structure when deployed into the heart valve can be configured to assume a conical shape such that the support structure has a smaller diameter at the one or more adjustable support beams than at the one or more non-adjustable circumferential rails.

In some cases, the support structure comprises one or more non-adjustable support beams and one or more adjustable circumferential rails. In some cases, a perimeter of the one or more adjustable circumferential rails is smaller than a perimeter of the heart valve during deployment of the support structure. The diameter of the one or more adjustable circumferential rails can be configured to increase after deployment of the support structure until the diameter of the one or more adjustable circumferential rails is equal to a diameter of the one or more non-adjustable support beams. The support structure deployed into the heart valve can be configured to generate a chronic force on the heart valve proportional to the difference between a perimeter of the support structure and a perimeter of the heart valve. In some embodiments, the perimeter of the support structure is equal to the perimeter of the heart valve such that the support structure when deployed into the heart valve does not generate a chronic force on the heart valve.

In some embodiments, the support structure comprises one or more adjustable support beams and one or more adjustable circumferential rails. In some cases, a perimeter of the one or more adjustable circumferential rails and a perimeter of the one or more adjustable support beams are smaller than a perimeter of the heart valve during insertion of the support structure. In some cases, the diameter of the one or more adjustable circumferential rails and the diameter of the one or more adjustable support beams are configured to increase after deployment of the support structure into the heart valve. The support structure when deployed can generate a chronic force proportional to the difference between a perimeter of the support structure and the perimeter of the heart valve after the increase of the diameter of the one or more adjustable circumferential rails and the diameter of the one or more adjustable support beams.

Disclosed herein is an intraluminal support structure for insertion into a heart valve of a subject, comprising a scaffold panel having a first free end and a second free end when the scaffold pattern is in a flattened configuration. The scaffold pattern in the flattened configuration further can comprise a first axis spanning from the first free end to the second free end. The first and second free ends can be configured to be coupled to each other to form into a ring when deployed. The second free end can comprise a cantilevered guiding slot. The first free end can comprise a hook that slidably couples to the cantilevered guiding slot in a first direction perpendicular to the first axis. In some cases, sliding the first free end to a terminal position in the first direction deflects the cantilevered guiding slot to retain the hook at the terminal position.

In some embodiments, the first free end and the second free end are parallel to each other and perpendicular to the first axis. In some cases, the second free end comprises one or more non-return features that restrict further sliding after the first free end and the second free end are slidably coupled. In some cases, the one or more non-return features activate after sliding the first free end to the terminal position and rotating the first free und until the hook enters the cantilevered guiding slot. The second free end can comprise one or more recoil springs. The second free end can comprise one or more latches.

In some cases, one or more of the first free end or the second free end are not perpendicular to the first axis.

Provided herein is a method for inserting an intraluminal support structure into a heart valve of a subject, comprising providing a scaffold panel having a first free end and a second free end when the scaffold pattern is in a flattened configuration. The scaffold pattern in the flattened configuration can further comprise a first axis spanning from the first free end to the second free end. The first and second free ends can be configured to be coupled to each other to form into a ring when deployed. The second free end can comprise a cantilevered guiding slot. The first free end can comprise a hook that slidably couples to the cantilevered guiding slot in a first direction perpendicular to the first axis. The method can further comprise sliding the first free end to a terminal position in the first direction into the cantilevered guiding slot. The method can further comprise loading the scaffold panel into a delivery sheath of a delivery catheter prior to sliding the first free end to a terminal position. In some cases, the method further comprises rotating the first free end of the scaffold panel counterclockwise prior to loading the scaffold panel into the delivery sheath. The method can further comprise rotating the first free end of the scaffold panel clockwise after sliding the first free end to a terminal position in the first direction.

Disclosed herein is a valve structure for insertion into a heart of a subject and configured to be coupled to an intraluminal support structure, comprising a scaffold panel having a first free end and a second free end when the scaffold panel is in a flattened configuration. The first and second free ends can be configured to be coupled to each other to form into a ring when deployed, transitioning the valve structure from a flattened configuration to a ring configuration. The valve structure can comprise one or more support bodies configured to form a central channel when the valve structure is in the ring configuration. The valve structure can comprise one or more central leaflet cusps. The valve structure can comprise one or more connecting skirts. In some cases, the one or more central leaflet cusps are inserted into an arch of the one or more valve support bodies. In some embodiments, the one or more connecting skirts are attached to the one or more valve support bodies.

In some cases, one or more of the one or more support bodies, the one or more central leaflet cusps, or the one or more connecting skirts are pre-assembled. The one or more support bodies can comprise reinforcement tabs configured to prevent commissural tearing. The one or more support bodies can comprise locking tabs configured to attach the one or more support bodies at a commissure top when the locking tabs are folded. The one or more connecting skirts can be configured to compensate for a diameter discrepancy between a central orifice of a heart valve and a diameter of the valve structure. The valve structure can comprise three sub-assemblies. In some cases, each sub-assembly comprises one support body, one leaflet cusp, and one connecting skirt. In some embodiments, one or more of the one or more support bodies, the one or more central leaflet cusps, or the one or more connecting skirts comprise biological tissue. In some cases, one or more of the one or more support bodies, the one or more central leaflet cusps, or the one or more connecting skirts comprise polymers. In some cases, one or more of the one or more support bodies, the one or more central leaflet cusps, or the one or more connecting skirts comprise hybrid tissue, including but not limited to a hybrid of biological tissue and polymers.

Provided herein is an intraluminal support structure for insertion into a heart valve of a subject, comprising a scaffold panel having a first free end and a second free end. The scaffold panel can be in a flattened configuration. The first and second free ends can be configured to be coupled to each other to form into a ring when deployed. The scaffold panel can further comprise a circumferential supporting portion spanning from the first free end to the second free end. The intraluminal support structure can comprise a plurality of gap filling arms extending from the circumferential supporting portion. In some cases, at least a terminal portion of the plurality of gap filling arms is parallel and offset from the circumferential supporting portion.

In some embodiments, the plurality of gap filling arms defines a cavity around the circumferential supporting portion. The cavity can have a round cross-section. The cavity can have a D-shaped cross-section. In some cases, the plurality of gap filling arms fills a void between the circumferential supporting portion and an anatomy of the heart valve. The plurality of gap filling arms can create a paravalvular leak sealing volume. The plurality of gap filling arms can dampen the force of the heart valve on the intraluminal support structure. The dampening effect of the plurality of gap filling arms can allow the intraluminal support structure to maintain a cylindrical valve orifice. The support structure can be non-deformable. The support structure can be deformable. In some cases, the intraluminal support structure is larger than the heart valve in an anterior-posterior direction. In some embodiments, the intraluminal support structure exerts force on the heart valve, wherein the gap filling arms are pushed outward. The gap filling arms can have an open toroidal braided structure. The open toroidal braided structure can comprise a nitinol or polymeric material.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the present disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1A is a schematic illustration of a heart showing the flow pattern therethrough and location for delivery of a prosthetic heart valve.

FIGS. 1B-1D depicts schematic, cross-sectional illustrations of the heart showing various state of the art trans-catheter approaches that allow a practitioner to reach the treatment area, namely, a native mitral valve. FIG. IB illustrates an antegrade approach wherein access is gained through the inter-atrial septum (IAS) maintained by the placement of a guide catheter over a guidewire FIG. 1C illustrates a retrograde approach to the native mitral valve. FIG ID illustrates a trans-apical approach to the mitral valve using a trans-apical puncture.

FIGS. 2A-2D depict planar views of an example embodiment of an intraluminal scaffold structure or stent as described herein. FIG. 2A shows the stent in a rolled out or flat configuration and FIGS. 2B-2D show components of the stent in magnified views.

FIGS. 2E-2F depict a perspective and top views of another example embodiment of an intraluminal scaffold structure or stent in a deployed configuration as described herein.

FIGS. 3A-3B depict schematic views of the force interactions between an intraluminal scaffold structure or stent as described herein and a native tissue of a patient.

FIGS. 4A-4C depict planar views of alternate example embodiments of intraluminal scaffold structures or stents as described herein.

FIG. 5 depicts a perspective view of the stent depicted in FIG. 4A in a deployed

FIGS. 6A-6C depict planar views of an example deformation mechanism of an intraluminal scaffold structure or stent as described herein. FIG. 6A shows the stent in a rolled out or flat configuration and FIGS. 6B-6C show the deformation mechanism of the stent in magnified views.

FIG. 7 depict a schematic view of the acting forces from and onto an example embodiment of an intraluminal scaffold structure or stent as described herein.

FIGS. 8A-8B depict schematic views of an example embodiment of an intraluminal scaffold structure or stent as described herein.

FIG. 8C depicts a perspective view of the stent as provided in the schematic views in FIGS. 8A-8B in a deployed configuration.

FIGS. 9A-9B depict schematic views of an example embodiment of an intraluminal scaffold structure or stent as described herein.

FIGS. 10A-10B depict side views of an example translation and deployment mechanism for an example embodiment of an intraluminal scaffold structure or stent as described herein.

FIGS. 11A-11C depict magnified side views of an example locking mechanism of an example embodiment of an intraluminal scaffold structure or stent as described herein.

FIG. 11D depicts a magnified perspective view of an example embodiment of a stent with the locking mechanism of FIGS. 11A-11C in a deployed configuration.

FIG. 12 depicts a planar view of an example embodiment of an intraluminal scaffold structure or stent in a rolled out or flattened configuration as described herein.

FIGS. 13A-13C depict magnified side views of an additional example locking mechanism of an example embodiment of an intraluminal scaffold structure or stent as described herein.

FIG. 13D depicts a perspective view of a stent with the locking mechanism of FIGS. 13A-13C in a deployed configuration.

FIGS. 14A-14I depict a bottom perspective (FIG. 14A), magnified side views (FIGS. 14B-14C), a bottom perspective view (FIG. 14D), a top perspective view (FIG. 14E), a side perspective view (FIG. 14F), a bottom perspective view (FIG. 14G), a top perspective view (FIG. 14H), and a side perspective view (FIG. 14I) of an alternate example of a deployment and locking mechanism for an example embodiment of an intraluminal scaffold structure or stent as described herein.

FIGS. 15A-15C depict magnified side (FIGS. 15A-15B) and magnified perspective (FIG. 15C) views of an additional example locking mechanism for an example embodiment of an intraluminal scaffold structure or stent as described herein.

FIGS. 16A-16C depict magnified side (FIGS. 16A-16B) and magnified perspective (FIG. 16C) views of another example locking mechanism for an example embodiment of an intraluminal scaffold structure or stent as described herein.

FIGS. 17A-17B depict schematic views of the expansion of an example embodiment of an intraluminal scaffold structure or stent as described herein once deployed into a human heart.

FIGS. 18-22 depict planar views of various example embodiments of intraluminal scaffold structure or stents in rolled or flattened configurations as described herein.

FIGS. 23A-23C depict a planar view (FIG. 23A), a perspective view (FIG. 23B), and a partial side view (FIG. 23C) of an example embodiment of an intraluminal scaffold structure or stent as described herein.

FIGS. 24A-24C depict a planar view (FIG. 24A) and perspective views (FIGS. 24B-24C) of another example embodiment of an intraluminal scaffold structure or stent in a rolled or flattened configuration (FIG. 24A) or in a deployed configuration (FIGS. 24B-24C) as described herein.

FIGS. 25A-25E depict views of examples of leaflet subunits for use in an example embodiment of a prosthetic valve as described herein. FIGS. 25A-25C depict side views, FIG. 25D depicts a top-down view, and FIG. 25E depicts a perspective view.

FIGS. 26A-26E depict various views of an example embodiment of a gap filling structure for intraluminal scaffold structures and stents as described herein. FIGS. 26A-26B depict planar views, FIG. 26C depicts a side section view, FIG. 26D shows a top view, and FIG. 26E shows a partial perspective view.

FIG. 27A depicts a perspective view of an example embodiment of an intraluminal scaffold structure or stent as described herein with an example embodiment of a gap filling structure as described herein.

FIGS. 27B-27C depict side and top schematic views, respectively, of the forces on an example embodiment of a stent as described herein with an example embodiment of a gap filling structure as described herein.

FIG. 28A depicts a perspective view of an alternate example embodiment of an intraluminal scaffold or stent as described herein with an example embodiment of a gap filling structure as described herein.

FIGS. 28B-28C depict side and top schematic views, respectively, of the forces on an alternate example embodiment of an intraluminal scaffold or stent as described herein with an example embodiment of a gap filling structure as described herein.

FIG. 29A depicts a side view of an intraluminal support scaffold or stent with an example embodiment of a gap filing structure as described herein. FIG. 29B depicts a side perspective view and a section view of the gap filling structure. FIG. 29C depicts a perspective view of an example embodiment of an intraluminal support scaffold or stent as described herein with the gap filling structure.

### DETAILED DESCRIPTION

The present disclosure relates generally to medical implants. More particularly, the present disclosure relates to devices for deployment into a human heart.

In Heart Valve Disease (HVD), a treatment mode can be replacement of the failing native valve with an artificial prosthetic valve. A prosthetic valve can provide a functional replacement of a damaged heart valve. A mode of delivery of placement of a prosthetic valve can be via catheterization techniques.

Catheter-based procedures can be used to treat patients suffering from failing native valve who are not candidates for open surgical procedure or wherein access with minimally invasive surgery is safer. In such catheter-based procedure, a stent or prosthetic valve may be delivered to a human heart using a percutaneous approach. In such delivery systems a prosthetic device, for example a stent or prosthetic valve or the like vessel support structures can be mounted on a balloon catheter or a self-expandable device that is advanced to the delivery site or location. At the delivery site a prosthetic device, for example a valvular device, is deployed by outwardly expanding the balloon from the distal end of the delivery system or retrieve the sheath in order to expand the self-expandable device. The balloon can be used to cause the prosthetic device to expand centrally, from the center out, so as to allow the deployment of the prosthetic device, for example a prosthetic valvular device, at the delivery site. Such central expansion can be limited for situations where large diameter valves are required, for example where the native valve annulus is extremely dilated. In such situations delivery systems become very large in diameter, limiting the possibility of navigation through the vasculature and the possibility of a trans-femoral trans-septal approach.

Delivery systems may be adapted according to the dimensions of the prosthetic device, balloon expandable device, or self-expandable device. In the case of atrioventricular (AV) valve replacements that use large valve devices, the apical approach may be used.

The left atrioventricular (AV valve) valve, also called the Mitral valve, poses unique anatomical obstacles, rendering percutaneous mitral valve replacement more challenging. The mitral valve's annulus has a non-circular D-shape or kidney-like shape, with a non-planar, saddle-like, geometry that can lacks symmetry. Such anatomical variation and non-symmetry makes it difficult to deliver a centrally expanding replacement valve utilizing a centrally expanding distal tip as is the current state of the art approach. In addition, anchoring the device by capturing the leaflets with hooks or loops can be hazardous and prone to failure.

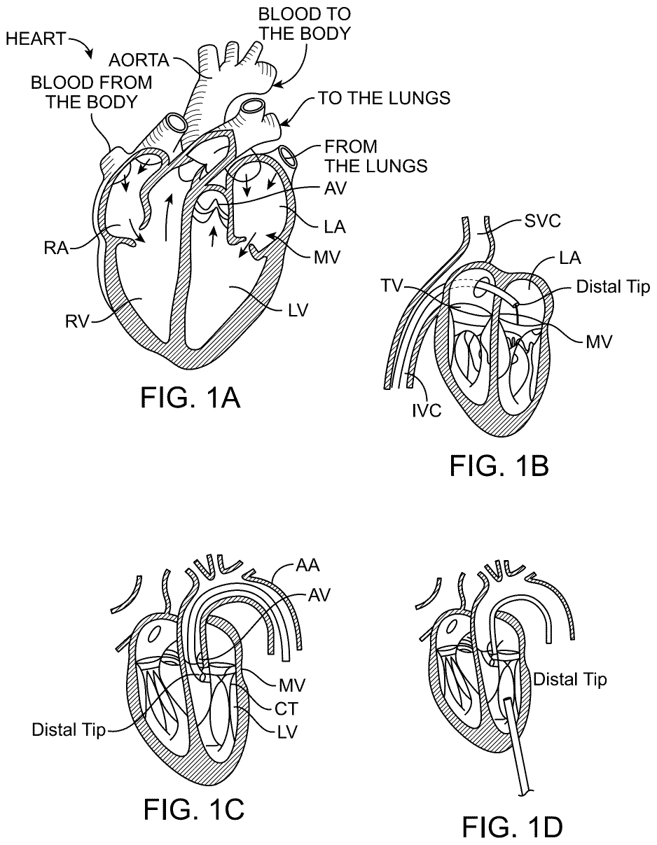

FIG. 1A shows the anatomy of a normal heart with the arrows depicting the flow through the valves. The heart comprises a left atrium (LA) that receives oxygenated blood from the lungs via the pulmonary veins (PV) and pumps this oxygenated blood through the mitral valve (MV) into the left ventricle (LV). When the left ventricle (LV) contracts, it allows blood to flow outwardly through the aortic valve (AV) in the direction of the arrows.

The mitral valve (MV) comprises a pair of leaflets that meet evenly, or “coapt” to close. The ventricular sides of the leaflets are attached to the surrounding heart structures of the left ventricle via an annular region of tissue referred to as the annulus (AN) found on the left atrium. The annulus is a fibrous ring of dense connective tissue which is distinct front both the leaflet tissue as well as the adjoining muscular tissue of the heart wall.

Access to the mitral valve or other atrioventricular valve can be accomplished through the patient's vasculature in a percutaneous manner. By “percutaneous” it is meant that a location of the vasculature remote from the heart is accessed through the skin. This can be done using a surgical cut down procedure or a minimally invasive procedure. Depending on the point of vascular access, the approach to the mitral valve may be antegrade and may rely on entry into the left atrium by crossing the inter-atrial septum, as shown in FIG. 1B. The FIG. 1B approach is also called the trans-septal approach. Alternatively, approach to the mitral valve can be retrograde where the left ventricle is entered through the aortic valve, for example as shown in FIG. 1C. An additional retrograde approach may be provided by a trans-apical puncture where access to the mitral valve is gained via a puncture at or near the apex of the heart, as shown in FIG. 1D.

Once access to the heart is achieved, the interventional tools and supporting catheter(s) may be advanced to the heart and positioned adjacent the target cardiac to allow for the deployment of the prosthetic devices. In such approaches, the functional end of the delivery system is found at the distal end or distal tip of the functional tools for example, catheter(s) and tool(s) used to deliver the prosthetic device(s).

Delivery systems can comprise a guidewire to introduce a guide catheter and the prosthetic device. After placing a guidewire, the guide catheter may be introduced over the guidewire to the treatment site.

### Basic Stent Forms

Described herein are prosthetic valve devices that include intraluminal scaffold structures or stents. These intraluminal scaffold or stents can comprise a plurality of elongate stent struts arranged into a fame or skeleton from which replacement valve leaflet structures are coupled, onto which allogenic tissue, synthetic tissue, or other coverings are overlaid, and/or onto which or other valve prosthesis structures, such as an atrial and/or ventricular sealing skirt, are coupled. Such prosthetic valve devices are configured for insertion or deployment into one or more heart valves, including the tricuspid valve, bicuspid or mitral valve, the pulmonary valve, or the aortic valve. The prosthetic valve devices described herein can be used to treat HVD or other heart diseases that improve with a transplant of a heart valve.

The intraluminal scaffold structures or stents of such prosthetic valve devices can be non-deformable. The stents can be deformable. The stents can comprise a variety of locking mechanisms. The stents can comprise various gap filling structures. The stent elements can be customizable based on the needs of a patient. The labeling of stents with different numbers does not indicate that the stents are only usable with the features they are used to demonstrate. Rather, any stent numbered or described herein can be used with any feature described herein.

The stent structures described herein have multiple benefits. They can provide and maintain a permanent cylindrical structure through an inter-locking mechanism by transitioning from an open flattened stent into a stiff cylinder. In some cases, they can maintain a high radial deformation resistance and crush resistance once implanted into a valve. In some cases, the stents can maintain a chronic radial force onto the valve once implanted. They can maintain a migration resistance under high pressure cardiac cycles. The stents can maintain a stiff support for the valvular components. The stents may fit the native anatomy and avoid flow obstruction once implanted.

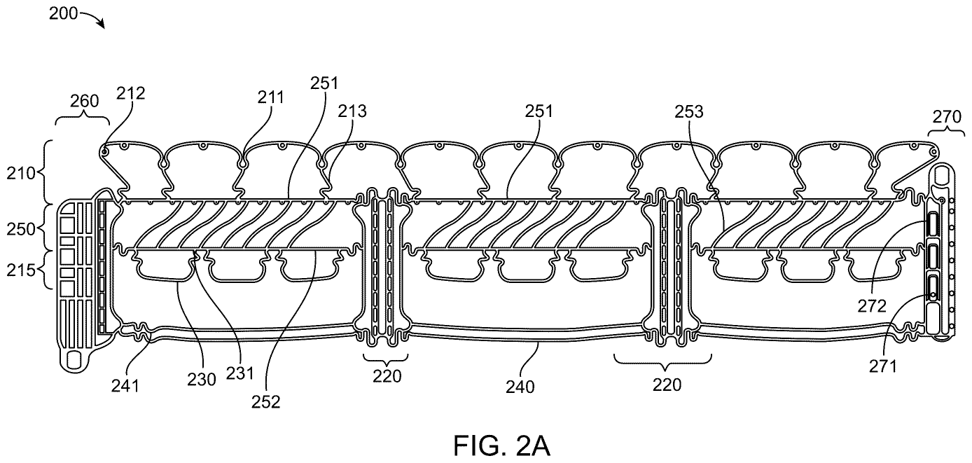

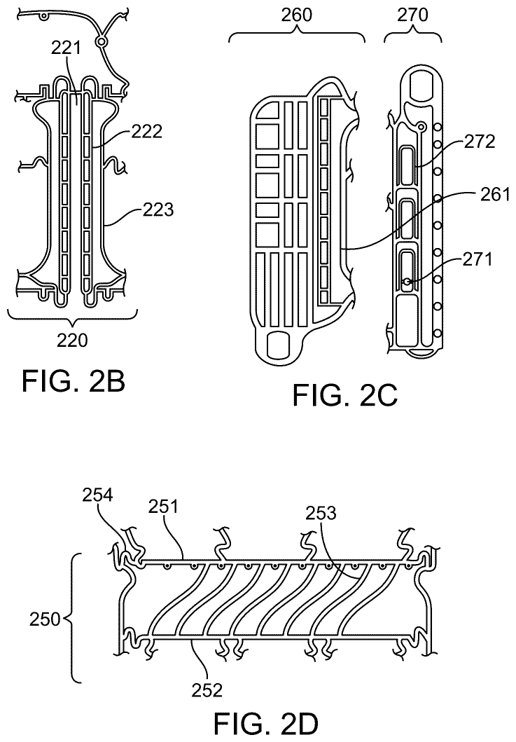

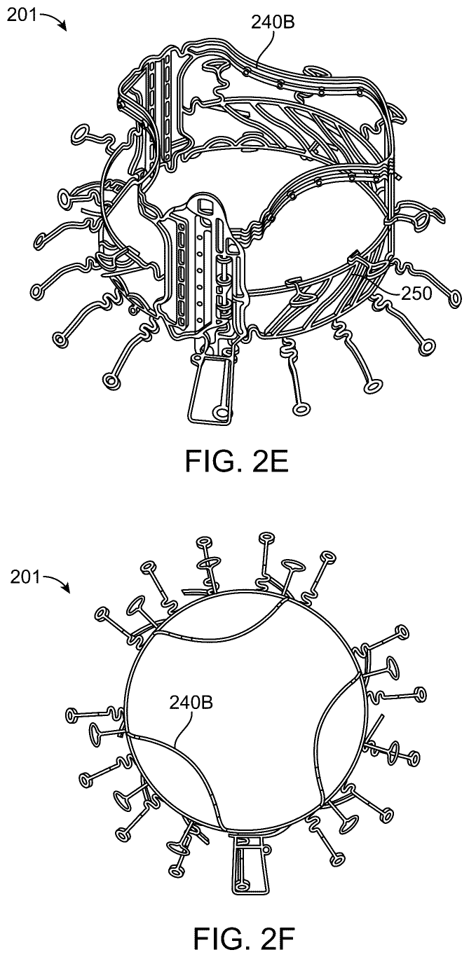

FIG. 2A depicts a planar view of an example stent structure 200. Stent 200 can comprise an upper (or superior or atrial) inflow crown 210 comprising crown deformation feature 211, fabric eyelet 212, crown dampener 213, lower (or inferior or ventricular) outflow crown 215, commissural stent post 220, flap 230, flap deformation feature 231, circumferential rail 240, rail strain relief 241, circumferential supporting beam 250, upper circumferential strut 251, lower circumferential strut 252, strut separator 253, proximal locking mechanism 260, distal locking mechanism 270, release wire eyelet 271, and locking hook 272.

Upper inflow crown 210 can be positioned on the atrial part of the stent structure. Once the two-dimensional (2D) planar stent structure is transitioned or deployed into a three-dimensional (3D) stent structure, upper inflow crown 210 sits externally to rest of the stent similar to a crown. Upper inflow crown 210 can be parallel to circumferential supporting beam 250. Upper inflow crown 210 can be parallel to circumferential rail 240. Upper inflow crown 210 can be perpendicular to commissural stent post 220. Upper inflow crown 210 can be perpendicular to proximal locking mechanism 260. Upper inflow crown 210 can be perpendicular to distal locking mechanism 270. Upper inflow crown 210 can be used to seal the stent against blood inflow during times of the cardiac cycle when the valve may otherwise be closed. Upper inflow crown 210 can comprise a plurality of vertical deformation features between horizontal connective features.

The stents described herein can comprise one or more deformation features, which are typically one or more crimped, sinusoidal, or otherwise looped or partially disconnected sections of an otherwise elongate stent strut. Such crimped or sinusoidal sections can allow the stent strut having such sections to lengthen or contract in its elongate direction (in other words, have a variable length) and dampen forces applied to the overall stent structure, for example, forces applied by the native heart valve structure and related anatomy from the heart beating. The one or more deformation features can also bias the overall stent structure to assume a desired shape when the stent is folded, rolled, or otherwise deployed into its deployed or three-dimensional (3D) configuration.

In some embodiments, a deformation feature can comprise a crown dampener 213. Crown dampener 213 can promote structural damping under cyclic loading of blood flow through heart valves. Structural damping by crown dampener 213 can promote stability of the stent once deployed into the valve. Structural damping by crown dampener 213 can promote migration resistance during valve deployment. Structural damping by crown dampener 213 can promote migration resistance once the stent is settled in the valve. Structural damping by crown dampener 213 can promote long term stability of the stent.

In some embodiments, an upper inflow crown 210 deformation feature can comprise a crown deformation feature 211. Crown deformation feature 211 can allow formation of the crown shape of the stent by stabilizing the longitudinal parts of the upper inflow crown 210.

In some cases, upper inflow crown 210 can comprise fabric eyelet 212. The stents described herein can be used in conjunction with heart valve fabrics. In some cases, fabric eyelet 212 can be used to attach heart valve fabrics to the stent, for example, by serving as apertures through which fabric or other materials can be stitched through.

FIG. 2B depicts a magnified view of commissural stent post 220 comprising central slots 221, commissural suturing feature 222, and vertical struts 223. Commissural stent post 220 can facilitate attachment of the pericardium leaflet or skirt to the stent. Pericardium can wrap around the commissural stent post. Commissural stent post 220 can prevent large commissural tip displacement. The commissural tip is the tip where the three commissures of a valve attach. Commissural stent post 220 can prevent interaction between native tissue leaflets and the prosthetic valve leaflets at the point of the circumferential rail.

Commissural stent post 220 can be perpendicular to circumferential supporting beam 250. Commissural stent post 220 can be perpendicular to circumferential rail 240. Commissural stent post 220 can be perpendicular to upper inflow crown. Commissural stent post 220 can be perpendicular to lower outflow crown. Commissural stent post 220 can be parallel to proximal locking mechanism 260. Commissural stent post 220 can be parallel to distal locking mechanism 270.

Commissural stent post 220 separates sections of circumferential rail 240 and lower outflow crown 215. Central slots 221 can receive commissural flaps. Vertical struts 223 can guide commissural flap attachment to the central slots 221. Vertical struts surrounding commissural suturing feature 222 can support commissural flaps. Suturing features 22 can support stability of the commissural stent post 220.

Commissural stent post 220 can comprise deformation features. Commissural flaps can open to allow crimping. Deformation features can promote damping under cyclic loading of blood flow through heart valves. Deformation features can promote damping. Structural damping by deformation features can promote stability of the stent once deployed into the valve. Structural damping by deformation features can promote migration resistance during valve deployment. Structural damping by deformation features can promote migration resistance once the stent is settled in the valve. Structural damping by deformation features can promote long term stability of the stent.

Stents as described herein can comprise a variety of locking mechanism. FIG. 2C, for example, comprises distal locking mechanism 270 comprising locking ratchet 272 and release wire eyelet 271, and proximal locking mechanism 260 comprising overlap zone 261. In some cases, stents can be deployed into the heart using a guidewire. In such cases, the guidewire can be inserted into the release wire eyelet 271. The distal locking mechanism 270 and proximal locking mechanism 260 can overlap in overlap zone 261 when the stent is in its deployed or 3D configuration. The joining of the distal locking mechanism 270 and proximal locking mechanism 260 can help the stent take on a 3D configuration. When they join, distal locking mechanism 270, proximal locking mechanism 260, or both may deflect from the circumference of the rest of the stent, thereby creating a commissure or joint.

Locking mechanisms can be perpendicular to circumferential supporting beam 250. Locking mechanisms can be perpendicular to circumferential rail 240. Locking mechanisms can be perpendicular to upper inflow crown. Locking mechanisms can be perpendicular to lower outflow crown. Locking mechanisms can be parallel to commissural stent post 220.

Stents as described herein can comprise a plurality of circumferential supporting beams. Circumferential supporting beams follow the circumference of a stent. In some cases, there is a single circumferential supporting beam following the entire circumference. In some embodiments, there are multiple circumferential supporting beams separated by commissural stent posts 220. In some embodiments, there are three circumferential supporting beams separated by commissural stent posts 220.

Stents as described herein can comprise a variety of circumferential supporting beams. For example, circumferential supporting beam 250 in FIG. 2D can comprise upper circumferential strut 251, lower circumferential strut 252, strut separators 253, and stress release features 251. Circumferential supporting beam 250 can comprise two or more parallel struts and a plurality of struts perpendicular to or angled relative to the two parallel struts. The parallel struts can comprise upper circumferential strut 251 and lower circumferential strut 252. The plurality of struts perpendicular to or angled relative to upper circumferential strut 251 and lower circumferential strut 252 comprise strut separators 253. Strut separators 253 can comprise deformation features. Strut separators 253 can be free of deformation features. Strut separators 253 can be straight, curved, angular, or any other design that connects the parallel struts.

Stress or strain release features 251 comprise crimping at the edges where the upper circumferential strut 251 and lower circumferential strut 252 meet the commissural stent posts 220. Stress or strain release features 251 can allow deformation of the stent without damaging or weakening the stent structure. This can allow loading of the stent into a heart of a patient without reaching structural plastic set as defined by Finite Element Analysis (FEA), for example, thus allowing the stent to maintain functionality after loading.

Circumferential supporting beam 250 can be parallel to upper inflow crown 210. Circumferential supporting beam 250 can be parallel to circumferential rail 240. Circumferential supporting beam 250 can be perpendicular to commissural stent post 220. Circumferential supporting beam 250 can be perpendicular to proximal locking mechanism 260. Circumferential supporting beam 250 can be perpendicular to distal locking mechanism 270.

Circumferential supporting beam 250 can provide circumferential stiffness to a stent as described herein. Circumferential supporting beam 250 can facilitate low radial deformation of a stent. Circumferential supporting beam 250 can help an inserted stent resist crushing forces of native tissue of the heart. Circumferential supporting beam 250 can define a sealing zone around the mitral annulus of the heart. Circumferential supporting beam 250 can support a sealing skirt of a stent as described herein by facilitating attachment of a valve skirt by protecting the skirt from migration and movement due to blood flow.

Stents as described herein can comprise a plurality of circumferential rails. Circumferential rails 240 follow the circumference or perimeter of a stent in its deployed or 3D configuration. In some cases, there is a single circumferential rail 240 following the entire circumference. In some embodiments, there are multiple circumferential rails 240 separated by commissural stent posts 220. In some embodiments, there are two circumferential rails 240 separated by commissural stent posts 220.

Circumferential rails 240 follow the circumference of a stent. In some cases, there is a single circumferential rail 240 following the entire circumference. In some embodiments, there are multiple sections of circumferential rails 240 separated by commissural stent posts 220. In some embodiments, there are three circumferential rail sections 240 separated by commissural stent posts 220. In some cases, each section of circumferential rail can comprise one or more struts. In some cases, each section of circumferential rail comprises two struts. Circumferential rail 240 can be the part of the stent furthest from upper inflow crown 210. Circumferential rail 240 can be parallel to upper inflow crown 210. Circumferential rail 240 can be parallel to lower outflow crown 215. Circumferential rail 240 can be parallel to circumferential supporting beam 250. Circumferential rail 240 can be perpendicular to commissural stent posts 220. Circumferential rail 240 can be perpendicular to proximal locking mechanism 260. Circumferential rail 240 can be perpendicular to distal locking mechanism 270. In some cases, circumferential rail 240 can limit commissural tip displacement. In some cases, circumferential rail 240 can prevent interaction between native tissue leaflets and stent valve leaflets by establishing a perimeter of a stent. In some embodiments, circumferential rail 240 can comprise rail strain relief feature 241. Rail strain relief feature 241 can be a small crimp near the edges of the circumferential rail 240 where it meets commissural stent posts 220.

In some cases, with reference to FIG. 2E, stents can comprise circumferential rail 240B. Circumferential rail 240B can comprise arcs towards the center of the stent. Circumferential rail 240B can comprise zero arcs. The arcs can be identical. The arcs can be similar. The arcs can be large or small. The arcs can be closely spaced or spaced farther. There can be between two to five arcs. There can be three arcs. The degree of arc can be between 90 degrees and 135 degrees. The degree of arc can be 120 degrees.

FIG. 2E shows a side view of a stent 201 with this design of the circumferential rail 240B. FIG. 2F shows a top-down view of a stent 201 with this design of the circumferential rail 240B. Circumferential rail 240B can function similarly to circumferential rail 240. Circumferential rail 240B can maintain commissural posts 220 separation. Circumferential rail 240B can prevent native tissue anatomy, for example chordae, from coming into contact with prosthetic tissue at the commissure. Circumferential rail 240B can reduce native tissue leaflet blockage with prosthetic leaflets in the outflow track. Circumferential rail 240B can stiffen the deflection of the commissural stent posts 220. Circumferential rail 240B can reduce protrusion of the stent structure into the blood outflow track.

Lower outflow crown 215 can be positioned directly below circumferential rail 240. Lower outflow crown 215 can be parallel to circumferential supporting beam 250. Lower outflow crown can be parallel to upper inflow crown 210. Lower outflow crown 215 can be perpendicular to commissural stent posts 220. Lower outflow crown 215 can be perpendicular to proximal locking mechanism 260. Lower outflow crown 215 can be perpendicular to distal locking mechanism 270. Lower outflow crown 215 can comprise flap 230 and flap deformation feature 231. Flaps 230 can capture native tissue valve leaflets. Lower outflow crown 215 can actuate sealing of the stent and valve by capturing the native tissue leaflets. By capturing the native leaflets, flaps 230 can promote migration resistance of the stent. In some embodiments, barbs can be added to the lower outflow crown to further resist migration of the stent when faced with strong migratory forces of circulating blood. As with crown deformation feature 211, flap deformation feature 231 can promote damping under cyclic loading of blood flow through heart valves. Structural damping by flap deformation feature 231 can promote stability of the stent once deployed into the valve. Structural damping by flap deformation feature 231 can promote migration resistance during valve deployment. Structural damping by flap deformation feature 231 can promote migration resistance once the stent is settled in the valve. Structural damping by flap deformation feature 231 can promote long term stability of the stent.

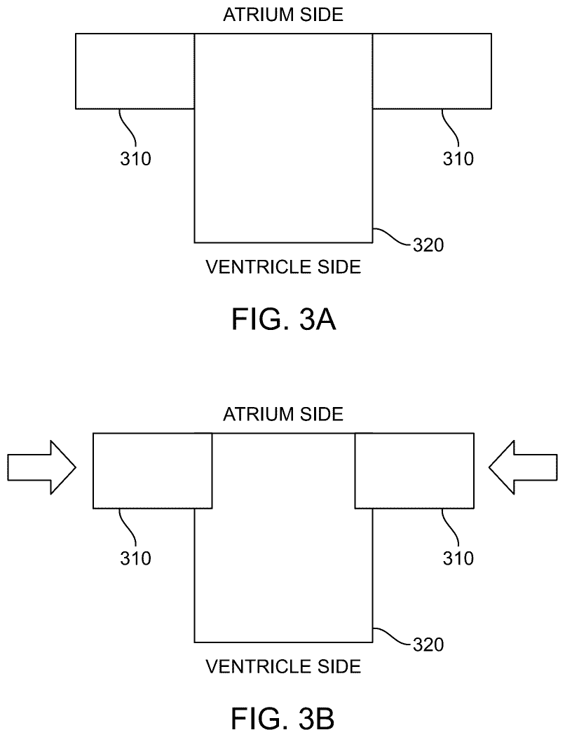

FIGS. 3A-3B display simplified, schematic versions of the implant structure comprising tissue anatomy 310 and stent implant 320.

FIG. 3A depicts a schematic view of the devices described herein wherein the implant or prosthesis oversize is zero percent. The diameter or perimeter or both of the stent implant 320 can be equal to the perimeter, diameter, or both of the mitral annulus. Stent implant 320 can remain cylindrical once implanted. The stent implant 320 may have zero chronic outward force onto the native heart anatomy. This can reduce the risk of mitral annulus rupture. Zero chronic outward force onto the native heart anatomy can also reduce the risk of electrophysiological conduction-related adverse events, including but not limited to heart block, arrhythmia, bundle branch block, long QT syndrome, and other similar heart issues.

FIG. 3B depicts a schematic view of the devices described herein wherein the implant oversize is greater than zero percent. Stent implant 320 can remain cylindrical once implanted. The diameter or perimeter or both of the stent implant 320 can be greater than the perimeter, diameter, or both of the mitral annulus. When the perimeter of the stent implant 320 is greater than the perimeter of the mitral annulus, the tissue anatomy can generate a reaction force proportional to the percentage oversize. This reaction force is depicted as arrows pointing inwards in FIG. 3B. With long-term implantation, there may be a degree of relaxation of the tissue anatomy as the tissue adjusts to the size of the stent implant 320. Relaxation or widening of the mitral annulus perimeter can reduce the risk of mitral annulus rupture. Relaxation or widening of the mitral annulus perimeter can also reduce the risk of electrophysiological conduction-related adverse events, including but not limited to heart block, arrhythmia, bundle branch block, long QT syndrome, and other similar heart issues. The deformation features provided with the prosthetic valve devices and their scaffold or stent structures or stents can allow such prosthetic valves to adjust accordingly to the relaxation or widening of the mitral valve annulus, providing, among other things, resistance to migration of the prosthetic valve device.

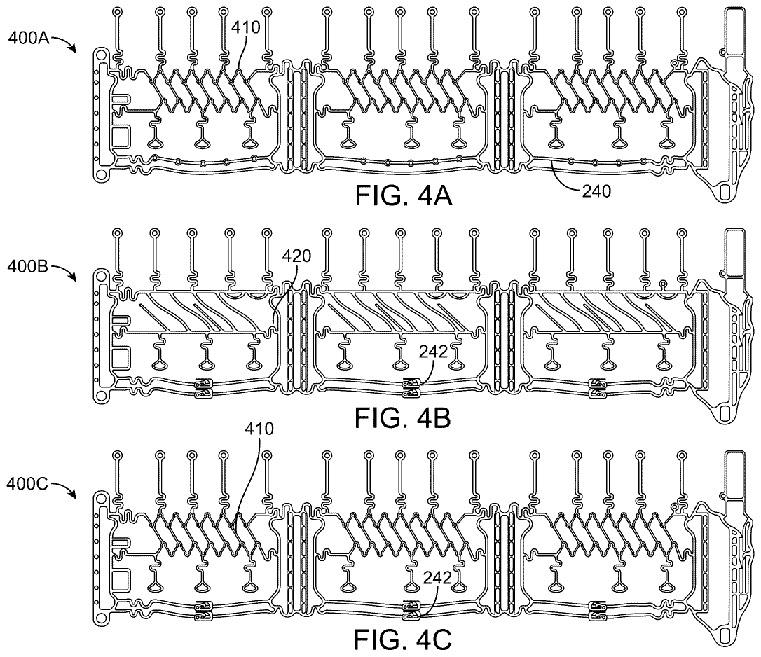

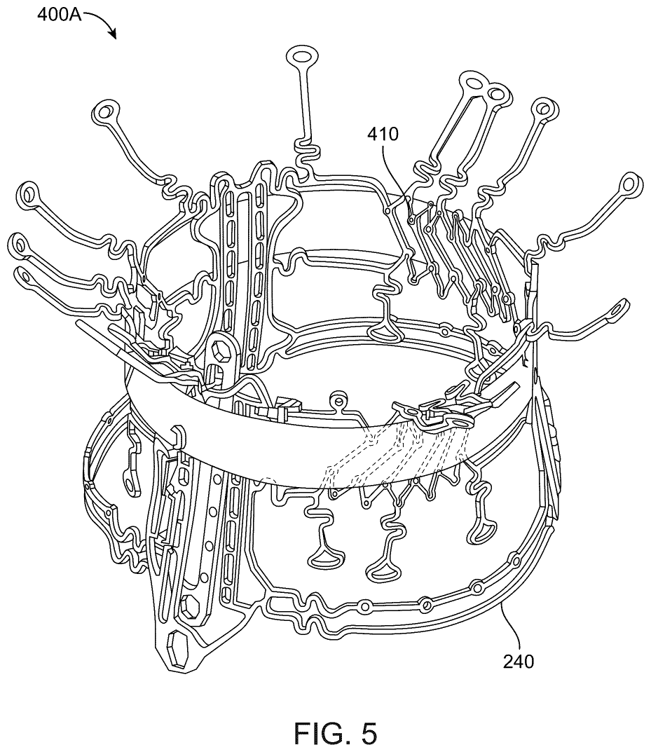

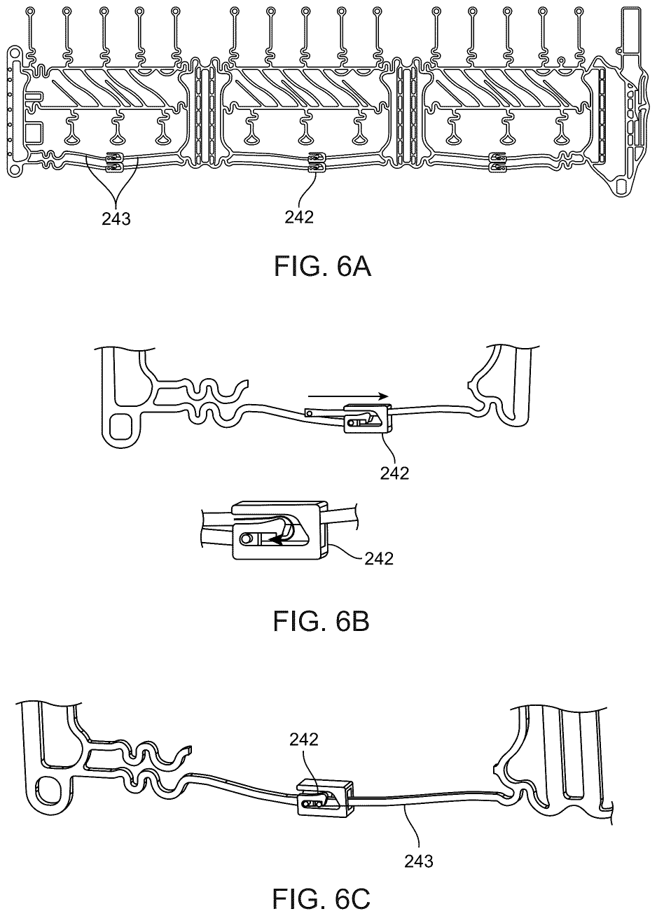

Stents as provided herein can comprise a variety of deformation features. Stents can comprise deformation features on the circumferential support beam. Examples of deformable circumferential support beams can be found in FIG. 4A, FIG. 4C, and FIG. 5. Stents can comprise deformation features on the circumferential rail, for example via a split frame circumferential rail with a variable locking mechanism. Examples of deformable circumferential rails can be found in FIG. 4B, FIG. 4C, and FIGS. 6A-6C.

FIGS. 4A-4C display three variations of deformable stents. In some cases, the upper inflow and lower outflow crowns comprise the crimped spikes ending in loops on either ends of the circumferential support beams. In some embodiments, an intraluminal support structure for deployment into a heart valve of a subject can comprise a scaffold panel having a first free end and a second free end when the scaffold panel is in a flattened configuration. The first and second free ends can be configured to be coupled to each other to form into a ring when deployed. The scaffold panel can comprise one or more support beams traversing the scaffold pattern between the first free end to the second free end at an atrial side of the scaffold panel. One or more circumferential rails can traverse the scaffold pattern between the first free end to the second free end at a ventricular side of the scaffold panel. A length of the one or more support beams, a length of the one or more circumferential rails, or both can be adjustable. In some cases, with reference to FIG. 4A, the intraluminal support structure can comprise one or more adjustable support beams and one or more non-adjustable circumferential rails. In some cases, an adjustable support beam comprises a deformable circumferential support beam.

In some cases, for example FIG. 4A, first deformable stent 400A can comprise deformable circumferential support beam 410 and circumferential rail 240.

Deformable circumferential support beam 410 can comprise quadrilaterals comprised of strut separators. Deformable circumferential support beam 410 can comprise rectangles, parallelograms, squares, rhombi, trapezoids, kites, or any other type of quadrilateral comprised of strut separators. In some embodiments, the areas where the strut separators meet form V-shaped cells. In some cases, deformable circumferential support beams do not comprise upper and lower circumferential struts. The long edges of the quadrilaterals comprise stiff connecting members. Quadrilateral strut separators can compress like an accordion at their V-shaped cells, giving them the ability to deform without permanently altering the shape of the stent. This ability can provide a stent structure with a variable perimeter length. The quadrilateral strut separators can comprise a propensity to return to their original perimeter, thus providing a chronic outward radial force while deformed.

In some cases, the quadrilateral strut separators can compress to less than 10%, less than 20%, less than 30%, less than 40%, less than 50%, less than 60%, less than 70%, less than 80%, less than 90%, or less than 100% of their full length.

Deformable circumferential support beam 410 can define a sealing zone around the mitral annulus. Deformable circumferential support beam 410 can support a sealing skirt of a stent as described herein by facilitating attachment of a valve skirt by protecting the skirt from migration and movement due to blood flow. The functionality of deformable circumferential support beam 410 can be similar to circumferential support beam 250.

First deformable stent 400A can comprise one or more circumferential rails. Circumferential rails 240 follow the circumference of a stent. In some cases, there is a single circumferential rail 240 following the entire circumference. In some embodiments, there are multiple sections of circumferential rails 240 separated by commissural stent posts 220. In some embodiments, there are three circumferential rail sections 240 separated by commissural stent posts 220. In some cases, each section of circumferential rail can comprise one or more struts. In some cases, each section of circumferential rail comprises two struts. Circumferential rail 240 can be the part of the stent furthest from upper inflow crown 210. Circumferential rail 240 can be parallel to upper inflow crown 210. Circumferential rail 240 can be parallel to lower outflow crown 215. Circumferential rail 240 can be parallel to circumferential supporting beam 250. Circumferential rail 240 can be perpendicular to commissural stent posts 220. Circumferential rail 240 can be perpendicular to proximal locking mechanism 260. Circumferential rail 240 can be perpendicular to distal locking mechanism 270. In some cases, circumferential rail 240 can limit commissural tip displacement. In some cases, circumferential rail 240 can prevent interaction between native tissue leaflets and stent valve leaflets by establishing a perimeter of a stent. In some embodiments, circumferential rail 240 can comprise rail strain relief feature 241. Rail strain relief feature 241 can be a small crimp near the edges of the circumferential rail 240 where it meets commissural stent posts 220.

FIG. 5 displays a three-dimensional view of first deformable stent 400A with deformable circumferential support beam 410 and circumferential rail 240.

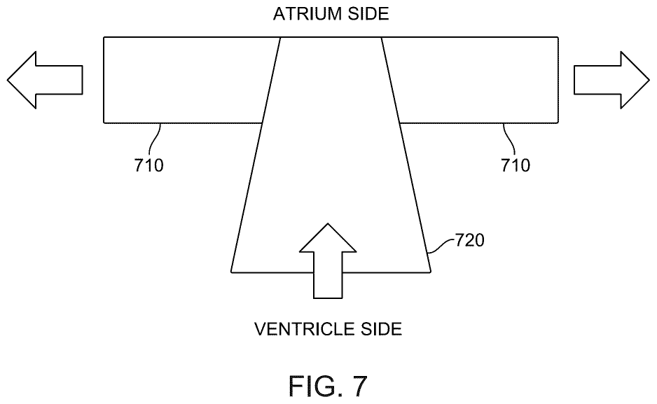

FIGS. 7A-7B depict schematic cross-sectional views of an implant 720, which can comprise the first deformable stent 400A, positioned with respect to tissue anatomy 710. The implant can comprise any of the embodiments of the deformable stents described herein. In some cases, implant 720 can comprise stent 400A. The arrow from the ventricular side to the atrium side can represent migration forces on a stent implant 720 due to systolic pressure once the stent is implanted in the heart. The arrows on either side of the anatomy 710 depict the direction of acting force from the stent implant 720 onto the anatomy 710. As the anatomy 710 surrounds the stent implant 720, an oversize of the stent implant 720 relative to the native anatomy 710 can generate force on the surrounding anatomy 710. The implant 720 can generate a chronic force proportional to the percentage oversize onto the anatomy 710.

In some embodiments, the perimeter of one of the adjustable support beams is less than the perimeter of the heart valve in which the intraluminal support structure is deployed. In some embodiments, the intraluminal support structure generates a chronic force proportional to the difference between a perimeter of the intraluminal support structure and the perimeter of the heart valve when deployed.

In some cases, with reference to when the implant 720 comprises stent 400A, the deformable circumferential supporting beam diameter or perimeter is smaller than the mitral annulus perimeter. In some cases, the circumferential support beam perimeter can be less than 10%, less than 20%, less than 30%, less than 40%, less than 50%, less than 60%, less than 70%, less than 80%, less than 90%, or less than 100% of the perimeter of the mitral annulus during deployment. The deformable circumferential supporting beam diameter or perimeter can be smaller than the diameter of the stent at the circumferential rail 240. In some cases, the circumferential support beam diameter or perimeter can be deformed to less than 10%, less than 20%, less than 30%, less than 40%, less than 50%, less than 60%, less than 70%, less than 80%, less than 90%, or less than 100% of the rail diameter. This can create a conical shape or any other shape where one part is wider than a second part.

In some cases, the intraluminal support structure when deployed into the heart valve is configured to assume a conical shape such that the intraluminal support structure has a smaller diameter at the one or more adjustable support beams than at the one or more non-adjustable circumferential rails. The different diameters of the stent, for instance, as adjusted over time, can provide additional migration resistance during a systolic pressure cycle. This is because the anatomy 710 can resist the larger diameter part of the stent.

FIG. 4B displays a second deformable stent 400B comprising non-deformable circumferential support beam 420 and circumferential rail adjuster 242. In some embodiments, with reference to stent 400B, the intraluminal support structure comprises one or more non-adjustable support beams and one or more adjustable circumferential rails.

Non-deformable circumferential support beam 420 is similar to circumferential support beam 250. Non-deformable circumferential support beam 420 can be parallel to the upper inflow crown. Non-deformable circumferential support beam 420 can be parallel to the circumferential rail. Non-deformable circumferential support beam 420 can be perpendicular to the commissural stent post. Non-deformable circumferential support beam 420 can be perpendicular to the proximal locking mechanism. Non-deformable circumferential support beam 420 can be perpendicular to the distal locking mechanism.

Non-deformable circumferential support beam 420 can provide circumferential stiffness to a stent as described herein. Non-deformable circumferential support beam 420 can facilitate low radial deformation of a stent. Non-deformable circumferential support beam 420 can help an inserted stent resist crushing forces of native tissue of the heart. Non-deformable circumferential support beam 420 can define a sealing zone around the mitral annulus of the heart. Non-deformable circumferential support beam 420 can support a sealing skirt of a stent as described herein by facilitating attachment of a valve skirt by protecting the skirt from migration and movement due to blood flow.

Second deformable stent 400B can comprise one or more circumferential rails. The circumferential rails are located in a similar place as in stent 200. Circumferential rails can follow the circumference of a stent. In some cases, there is a single circumferential rail following the entire circumference. In some embodiments, there are multiple section of circumferential rails separated by commissural stent posts. In some embodiments, there are three circumferential rail sections separated by commissural stent posts. In some cases, each section of circumferential rail can comprise one or more struts. In some cases, each section of circumferential rail comprises two struts. In some embodiments, the circumferential rail can comprise rail strain relief feature. A rail strain relief feature can be a small crimp near the edges of the circumferential rail where it meets the commissural stent posts.

In addition to or in place of the crimping methods described above for deforming a circumferential rail, the circumferential rail adjuster 242 can be used to deform the circumferential rail. Deformation features on the circumferential rail can comprise a variable length system. A variable length system can comprise a variable length locking mechanism as shown in FIGS. 6A-6C.

FIG. 6A comprises a split circumferential rail 243 and circumferential rail adjuster 242. FIG. 6B and FIG. 6C display magnified views of how circumferential rail adjuster 242 moves along and locks split circumferential rail 243, thus acting as a variable length locking mechanism. While being locked to one side of the split circumferential rail 243, circumferential rail adjuster moves towards the other side of the split circumferential rail in an open position. In the open position, the second side of the split circumferential rail can pass through an opening in circumferential rail adjuster 242. When the stent perimeter reaches the predetermined perimeter, the circumferential rail adjuster 242 can be changed into a locked position to hold the second side of the split circumferential rail. This way, after movement and locking, the first half of the split circumferential rail retains its full length while the second half of the split circumferential rail can be smaller than its full length. In some cases, the second half of the split circumferential rail comprises its full length. In some cases, the second half of the split circumferential rail comprises greater than 10%, greater than 20%, greater than 30%, greater than 40%, greater than 50%, greater than 60%, greater than 70%, greater than 80%, or greater than 90% less than its full length. In some cases, the second half of the split circumferential rail comprises less than 10%, less than 20%, less than 30%, less than 40%, less than 50%, less than 60%, less than 70%, less than 80%, or less than 90% less than its full length.

A variable or deformable circumferential rail can reduce the diameter of the stent. A variable or deformable circumferential rail can reduce the diameter of the stent to customize the stent for the heart into which it is inserted. A variable or deformable circumferential rail can reduce the diameter of the stent after insertion based on the needs of the patient. A variable or deformable circumferential rail can reduce the diameter of the stent during insertion for easier insertion. Once implanted, a variable or deformable circumferential rail can exert chronic radial force onto the native tissue. The functionality of deformable circumferential support beams can be similar to circumferential rail 240 or 240B, including but not limited to decreasing interaction between native tissue leaflets and the stent valve leaflets, and limiting displacement of the commissural tip.

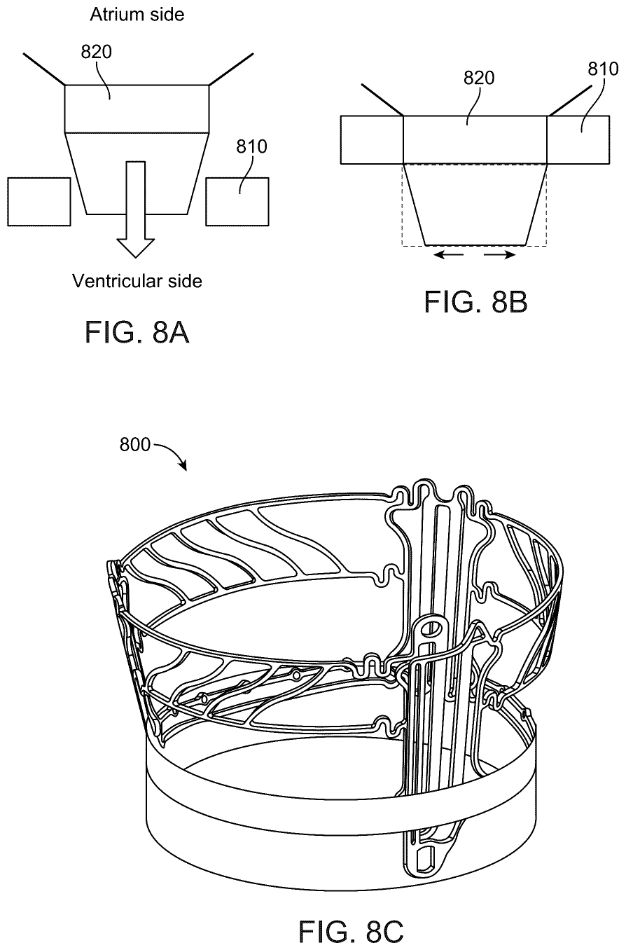

FIGS. 8A and 8B display schematic cross-sectional views of one version of second deformable stent 400B comprising tissue anatomy 810 and implant 820. The implant can comprise any of the embodiments of the deformable stents described herein. The arrow from the atrium side to the ventricular side in FIG. 8A depicts a possible direction of insertion of implant 820 into tissue anatomy 810.

In some cases, with reference to stent 400B, the deformable circumferential rail diameter or perimeter can be smaller than the mitral annulus perimeter during deployment. In some cases, the circumferential rail perimeter can be less than 10%, less than 20%, less than 30%, less than 40%, less than 50%, less than 60%, less than 70%, less than 80%, less than 90%, or less than 100% of the perimeter of the mitral annulus during deployment. In some cases, the circumferential rail diameter or perimeter can be deformed to less than 10%, less than 20%, less than 30%, less than 40%, less than 50%, less than 60%, less than 70%, less than 80%, less than 90%, or less than 100% of their full length. This can create a conical shape or any other shape where one part is wider than a second part. A non-schematic view of a conical shape of a stent as described herein 800 with a deformable circumferential rail is shown in FIG. 8C. With a smaller circumferential rail diameter, the stent can be lowered into anatomy 810 with little interference. After insertion, the deformable circumferential rail can be re-expanded to its full diameter and perimeter by mechanical mechanisms, as shown in FIG. 8B. After expansion, deformable circumferential rail can regain the same diameter as non-deformable circumferential support beam 420. In some embodiments, the diameter of the one or more adjustable circumferential rails is configured to increase after deployment of the intraluminal support structure until the diameter of the one or more adjustable circumferential rails is equal to a diameter of the one or more non-adjustable support beams.

In some cases, after insertion implant 820 can comprise zero percent oversize or greater than zero percent oversize relative to the native anatomy.

In some embodiments, where the implant oversize is zero percent, the diameter or perimeter or both of the stent implant 820 can be equal to the perimeter, diameter, or both of the mitral annulus. In some embodiments, the perimeter of the intraluminal support structure is equal to the perimeter of the heart valve such that the intraluminal support structure when deployed into the heart valve does not generate a chronic force on the heart valve. Stent implant 820 can remain cylindrical once implanted. The stent implant 820 may have zero chronic outward force onto the native heart anatomy. This can reduce the risk of mitral annulus rupture. Zero chronic outward force onto the native heart anatomy can also reduce the risk of electrophysiological conduction-related adverse events, including but not limited to heart block, arrhythmia, bundle branch block, long QT syndrome, and other similar heart issues.

In some cases, where the implant oversize is greater than zero percent, stent implant 820 can remain cylindrical once implanted. The diameter or perimeter or both of the stent implant 820 can be greater than the perimeter, diameter, or both of the mitral annulus. When the perimeter of the stent implant 820 is greater than the perimeter of the mitral annulus, the tissue anatomy can generate a reaction force proportional to the percentage oversize. This reaction force is depicted as arrows pointing inwards. With long-term implantation, there may be a degree of relaxation of the tissue anatomy as the tissue adjusts to the size of the stent implant 820. Relaxation or widening of the mitral annulus perimeter can reduce the risk of mitral annulus rupture. Relaxation or widening of the mitral annulus perimeter can also reduce the risk of electrophysiological conduction-related adverse events, including but not limited to heart block, arrhythmia, bundle branch block, long QT syndrome, and other similar heart issues.

In some embodiments, the perimeter of one of adjustable circumferential rails is less than the perimeter of the heart valve in which the intraluminal support structure is deployed. In some embodiments, the intraluminal support structure generates a chronic force proportional to the difference between a perimeter of the intraluminal support structure and the perimeter of the heart valve when deployed.

FIG. 4C depicts a third deformable stent 400C comprising deformable circumferential support beam 410 and circumferential rail adjuster 242. The circumferential rail adjuster 242 can be used to deform the circumferential rail. In some cases, the intraluminal support structure comprises one or more adjustable support beams and one or more adjustable circumferential rails.

Deformable circumferential support beam 410 can comprise quadrilaterals comprised of strut separators. Deformable circumferential support beam 410 can comprise rectangles, parallelograms, squares, rhombi, trapezoids, kites, or any other type of quadrilateral comprised of strut separators. In some embodiments, the areas where the strut separators meet form V-shaped cells. In some cases, deformable circumferential support beams do not comprise upper and lower circumferential struts. The long edges of the quadrilaterals comprise stiff connecting members. Quadrilateral strut separators can compress like an accordion at their V-shaped cells, giving them the ability to deform without permanently altering the shape of the stent. This can provide a stent structure with a variable perimeter length. The quadrilateral strut separators can comprise a propensity to return to their original perimeter, thus providing a chronic outward radial force while deformed.

In some cases, the quadrilateral strut separators can compress to less than 10%, less than 20%, less than 30%, less than 40%, less than 50%, less than 60%, less than 70%, less than 80%, less than 90%, or less than 100% of their full length.

Deformable circumferential support beam 410 can define a sealing zone around the mitral annulus. Deformable circumferential support beam 410 can support a sealing skirt of a stent as described herein by facilitating attachment of a valve skirt by protecting the skirt from migration and movement due to blood flow. The functionality of deformable circumferential support beam 410 can be similar to circumferential support beam 250.

Third deformable stent 400C can comprise one or more circumferential rails. The circumferential rails are located in a similar place as in stent 200. Circumferential rails can follow the circumference of a stent. In some cases, there is a single circumferential rail following the entire circumference. In some embodiments, there are multiple section of circumferential rails separated by commissural stent posts. In some embodiments, there are three circumferential rail sections separated by commissural stent posts. In some cases, each section of circumferential rail can comprise one or more struts. In some cases, each section of circumferential rail comprises two struts. In some embodiments, the circumferential rail can comprise rail strain relief feature. A rail strain relief feature can be a small crimp near the edges of the circumferential rail where it meets the commissural stent posts.

In addition to or in place of the crimping methods described above for deforming a circumferential rail, the circumferential rail adjuster 242 can be used to deform the circumferential rail. Deformation features on the circumferential rail can comprise a variable length system. A variable length system can comprise a variable length locking mechanism.

Circumferential rail adjuster 242 moves along and locks a split circumferential rail, thus acting as a variable length locking mechanism. While being locked to one side of the split circumferential rail, the circumferential rail adjuster moves towards the other side of the split circumferential rail in an open position. In the open position, the second side of the split circumferential rail can pass through an opening in the circumferential rail adjuster. When the stent perimeter reaches the desired perimeter, the circumferential rail adjuster can be changed into a locked position to hold the second side of the split circumferential rail. This way, after movement and locking, the first half of the split circumferential rail retains its full length while the second half of the split circumferential rail can be smaller than its full length. In some cases, the second half of the split circumferential rail comprises its full length. In some cases, the second half of the split circumferential rail comprises greater than 10%, greater than 20%, greater than 30%, greater than 40%, greater than 50%, greater than 60%, greater than 70%, greater than 80%, or greater than 90% less than its full length. In some cases, the second half of the split circumferential rail comprises less than 10%, less than 20%, less than 30%, less than 40%, less than 50%, less than 60%, less than 70%, less than 80%, or less than 90% less than its full length.

A variable or deformable circumferential rail can reduce the diameter of the stent. A variable or deformable circumferential rail can reduce the diameter of the stent to customize the stent for the heart into which it is inserted. A variable or deformable circumferential rail can reduce the diameter of the stent after insertion based on the needs of the patient. A variable or deformable circumferential rail can reduce the diameter of the stent during insertion for easier insertion. Once implanted, a variable or deformable circumferential rail can exert chronic radial force onto the native tissue. The functionality of deformable circumferential support beams can be similar to circumferential rail 240 or 240B, including but not limited to decreasing interaction between native tissue leaflets and the stent valve leaflets, and limiting displacement of the commissural tip.

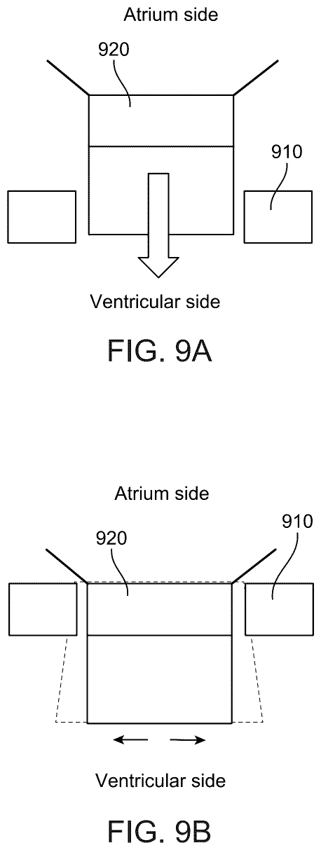

FIGS. 9A-9B display schematic cross-sectional views of one version of third deformable stent 400C comprising tissue anatomy 910 and implant 920. The implant can comprise any of the embodiments of the deformable stents described herein. In some cases, implant 920 can comprise stent 400C. The arrow from the atrium side to the ventricular side shows a possible direction of insertion of implant 920 into tissue anatomy 910, as shown in FIG. 9A. The arrows pointing left and right in FIG. 9B represent a release of the deformation of the circumferential support beam and circumferential rail, thereby expanding the stent implant 920.

In some embodiments, a perimeter of the one or more adjustable circumferential rails and a perimeter of the one or more adjustable support beams are smaller than a perimeter of the heart valve during insertion of the intraluminal support structure. In some cases, the diameter of the one or more adjustable circumferential rails and the diameter of the one or more adjustable support beams are configured to increase after deployment of the intraluminal support structure into the heart valve.

In some cases, with reference to stent 400C, the deformable circumferential rail diameter or perimeter can be smaller than the mitral annulus perimeter during deployment. In some cases, the circumferential rail perimeter can be less than 10%, less than 20%, less than 30%, less than 40%, less than 50%, less than 60%, less than 70%, less than 80%, less than 90%, or less than 100% of the perimeter of the mitral annulus during deployment. In some cases, the circumferential rail diameter or perimeter can be deformed to less than 10%, less than 20%, less than 30%, less than 40%, less than 50%, less than 60%, less than 70%, less than 80%, less than 90%, or less than 100% of their full length. With a smaller circumferential rail diameter, the stent can be lowered into anatomy 910 with little interference. After insertion, the deformable circumferential rail can be re-expanded to its full diameter and perimeter by mechanical mechanisms, as shown in FIG. 9B.

In some cases, with reference to stent 400C, the deformable circumferential support beam diameter or perimeter can be smaller than the mitral annulus perimeter during deployment. In some cases, the circumferential support beam perimeter can be less than 10%, less than 20%, less than 30%, less than 40%, less than 50%, less than 60%, less than 70%, less than 80%, less than 90%, or less than 100% of the perimeter of the mitral annulus during deployment. In some cases, the circumferential support beam diameter or perimeter can be deformed to less than 10%, less than 20%, less than 30%, less than 40%, less than 50%, less than 60%, less than 70%, less than 80%, less than 90%, or less than 100% of their full length. With a smaller circumferential support beam diameter, the stent can be lowered into anatomy 910 with little interference. After insertion, the deformable circumferential support beam can be re-expanded to its full diameter and perimeter by mechanical mechanisms, as shown in FIG. 9B.

In some embodiments, the intraluminal support structure when deployed generate a chronic force proportional to the difference between a perimeter of the support structure and the perimeter of the heart valve after the increase of the perimeter of the one or more adjustable circumferential rails and the perimeter of the one or more adjustable support beams. In some cases, once implanted, third deformation stent 400C acts like implant 720. As the anatomy 710 surrounds the stent implant 720, an oversize of the stent implant 720 relative to native anatomy 710 can generate force on the surrounding anatomy 710. The implant 720 can generate a chronic force proportional to the percentage oversize onto the anatomy 710. Although both the deformable circumferential support beam and deformable circumferential rail can expand, in some cases the deformable circumferential rail expands more than deformable circumferential support beam. In some embodiments, deformable circumferential rail can expand to its maximum perimeter while deformable circumferential support beam. In some cases, the circumferential support beam diameter or perimeter can be deformed to less than 10%, less than 20%, less than 30%, less than 40%, less than 50%, less than 60%, less than 70%, less than 80%, less than 90%, or less than 100% of the rail diameter. This deformation can create a conical shape or any other shape where one part is wider than a second part. The different diameters of the stent can provide additional migration resistance during a systolic pressure cycle, because the anatomy 710 can resist part of the stent with the larger diameter.

### Locking Mechanisms and Methods

Provided herein are locking mechanisms and methods to be used with one or more of the stents described herein. Described herein are intraluminal support structures for insertion into a heart valve of a subject, comprising a scaffold panel having a first free end and a second free end when the scaffold pattern is in a flattened configuration. The scaffold pattern in the flattened configuration can further comprise a first axis spanning from the first free end to the second free end. The first and second free ends can be configured to be coupled to each other to form into a ring when deployed. The second free end can comprise a cantilevered guiding slot. The first free end can comprise a hook that slidably couples to the cantilevered guiding slot in a first direction perpendicular to the first axis. sliding the first free end to a terminal position in the first direction deflects the cantilevered guiding slot to retain the hook at the terminal position.

In some cases, the first free end and the second free end are parallel to each other and perpendicular to the first axis. In some embodiments, the second free end comprises one or more non-return features that restrict further sliding after the first free end and the second free end are slidably coupled. The one or more non-return features can activate after sliding the first free end to the terminal position and rotating the first free und until the hook enters the cantilevered guiding slot. The second free end can comprise one or more recoil springs. The second free end can comprise one or more latches.

In some cases, one or more of the first free end or the second free end are not perpendicular to the first axis. There can be an angle between the main axis of the stent and the locking mechanism.

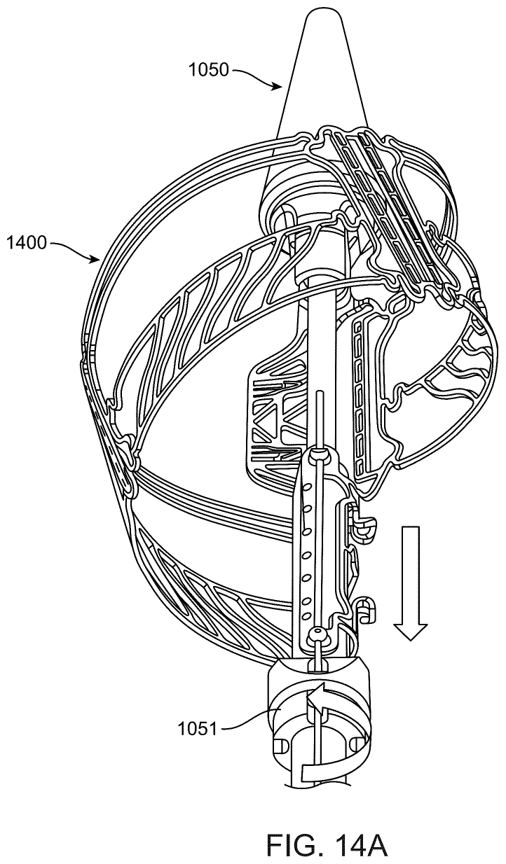

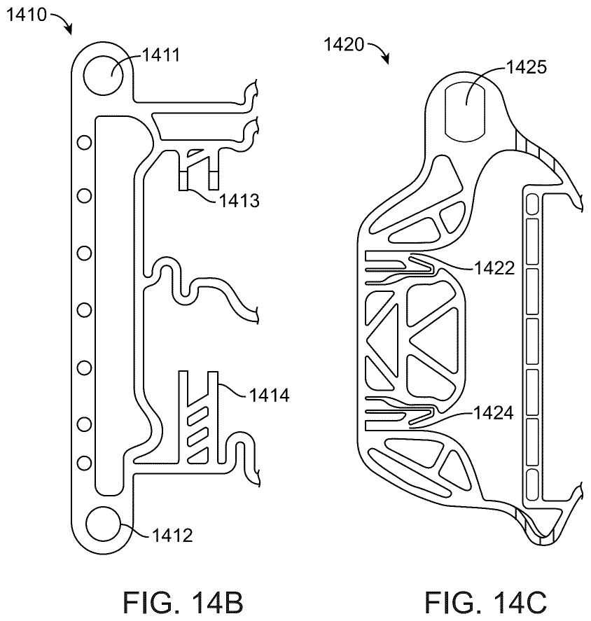

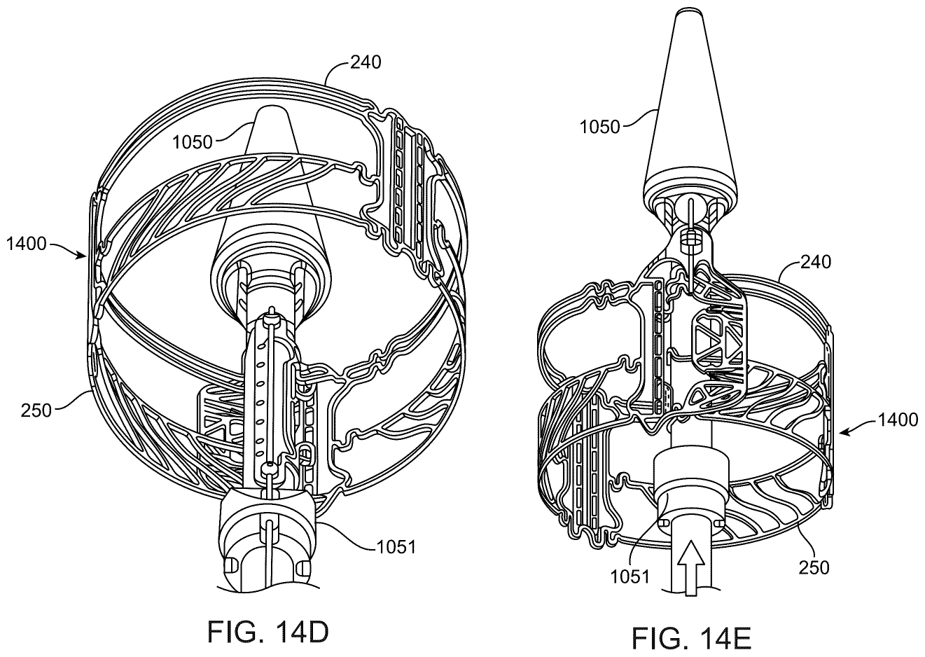

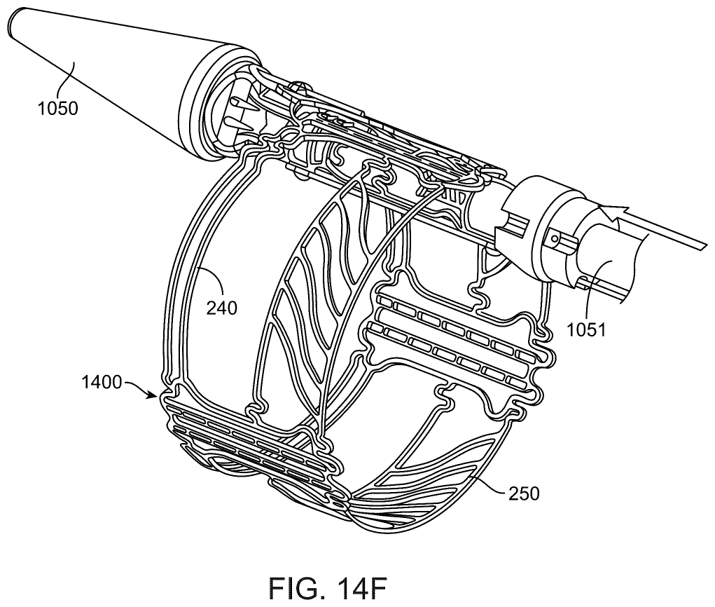

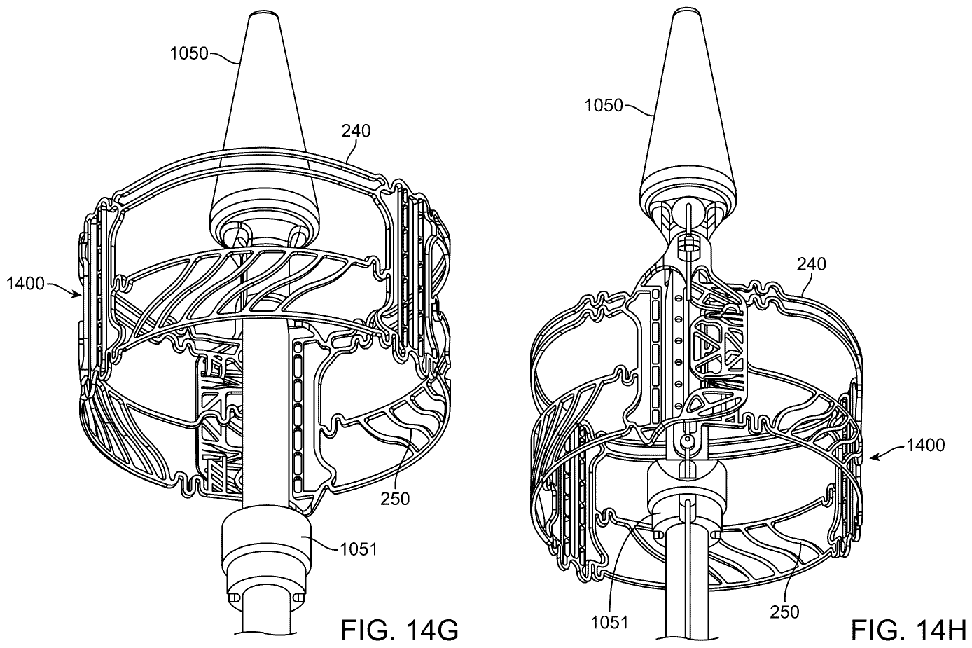

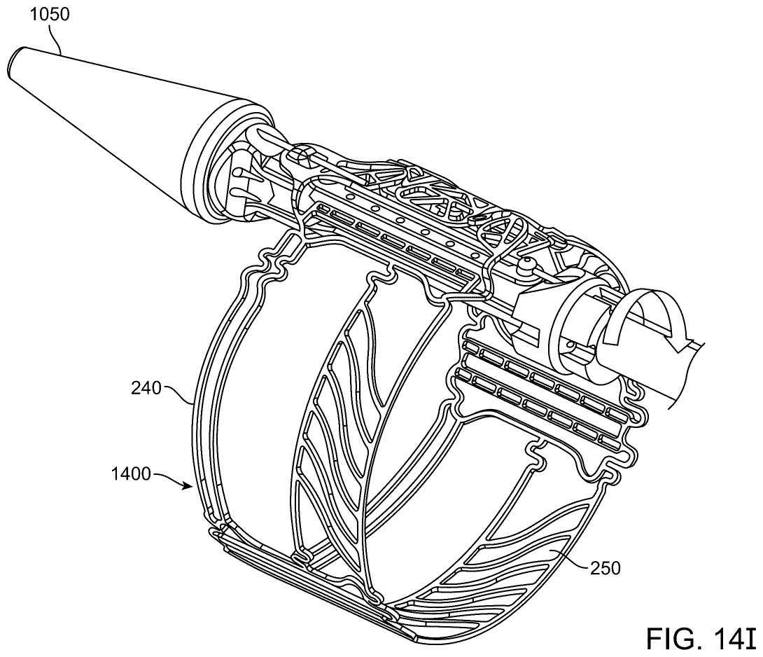

Described herein are methods for deploying an intraluminal support structure into a heart valve of a subject, comprising providing a scaffold panel having a first free end and a second free end when the scaffold pattern is in a flattened configuration. The scaffold pattern in the flattened configuration can further comprise a first axis spanning from the first free end to the second free end. The first and second free ends can be configured to be coupled to each other to form into a ring when deployed. the second free end can comprise a cantilevered guiding slot. The first free end can comprise a hook that slidably couples to the cantilevered guiding slot in a first direction perpendicular to the first axis. Sliding the first free end to a terminal position in the first direction into the cantilevered guiding slot. In some cases, the method can further comprise loading the scaffold panel into a delivery sheath of a delivery catheter prior to sliding the first free end to a terminal position. In some embodiments, the method can further comprise rotating the first free end of the scaffold panel counterclockwise prior to loading the scaffold panel into the delivery sheath. The method can further comprise rotating the first free end of the scaffold panel clockwise after sliding the first free end to a terminal position in the first direction. An example of this rotation can be seen with the radial closing mechanism displayed in FIG. 14.

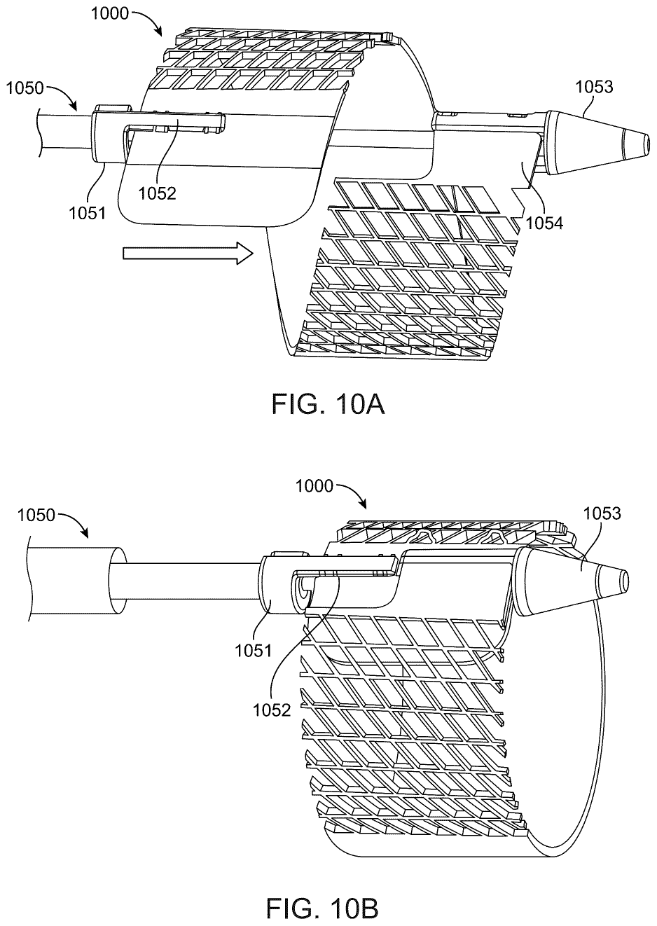

FIGS. 10A-10B depict an axial closing mechanism comprising delivery catheter 1050 and a stent as described herein 1000. This closing mechanism can be used with one or more of the distal or proximal locking elements described herein. FIG. 10A shows the starting position pre-translation of the delivery catheter 1050 comprising mobile proximal portion 1051, proximal attachment area 1052, fixed distal nose cone 1053, and distal attachment area 1054. The arrow shows the direction of translation. Mobile proximal portion 1051 is located on the atrium side of the stent, whereas the fixed distal nose cone 1053 is located on the ventricular side of the stent.

FIG. 10B displays the ending position in a locked position. The mobile proximal portion 1051 of the delivery catheter 1050 holding the proximal end of a stent 1000 with a proximal attachment area 1052 can slide or translate towards the fixed distal nose cone 1053 of the delivery catheter 1050 holding the distal end of a stent 1000 with distal attachment area 1054. This mechanism can be used to turn a planar flattened configuration of a stent into a cylindrical or ring structure. The cylinder can be stiff. The stiffness and locking of the stent 1000 can be used to maintain a permanent cylindrical structure of the stent 1000 once deployed into the heart. In some cases, the locking system removes all degrees of freedom. In some cases, the locking system removes greater than almost all degrees of freedom.

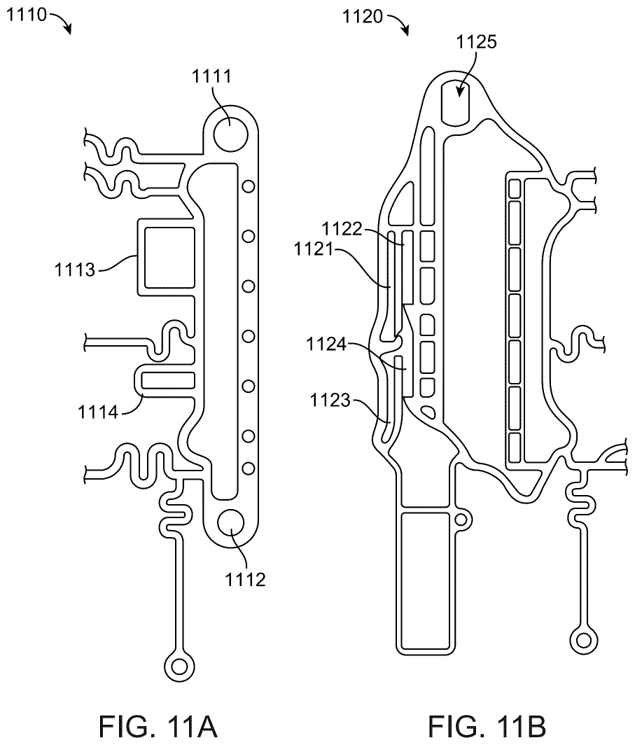

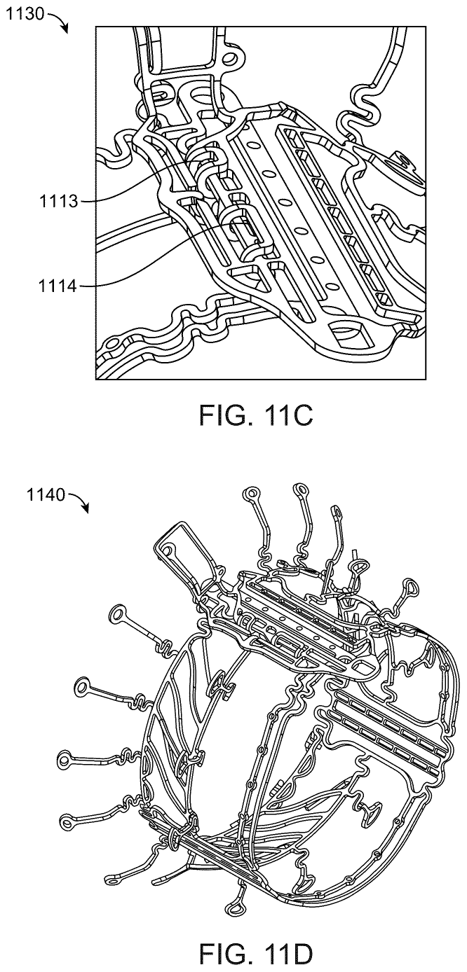

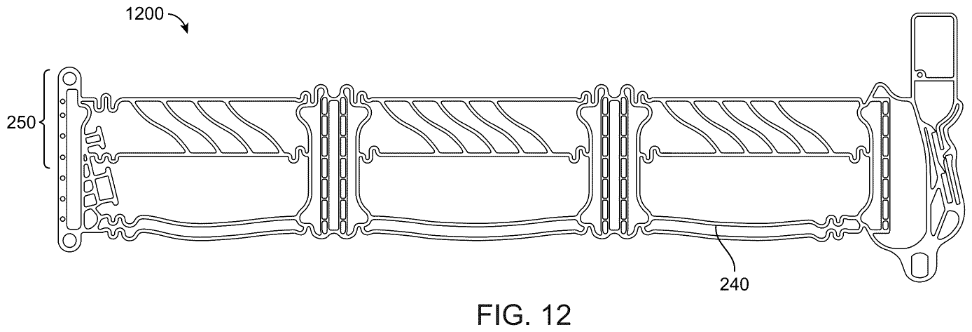

FIG. 11A depicts the insertable attachment feature 1110 of the locking mechanism comprising upper proximal attachment feature 1111, lower proximal attachment feature 1112, upper hook 1113, and lower hook 1114. The upper proximal attachment feature 1111 and lower proximal attachment feature 1112 are the most proximal elements of a flattened planar view of a stent as described herein. Upper hook 1113 and lower hook 1114 can be inserted into the receiving attachment feature 1120 upon reaching them through the translation facilitated by the delivery catheter 1050 as described above.

FIG. 11B depicts the receiving attachment feature 1120 of the locking mechanism comprising upper retaining latch 1121, upper cantilevered guiding slot 1122, lower retaining latch 1123, lower cantilevered guiding slot 1124, and upper distal attachment feature 1125. Receiving attachment feature 1120 is the most distal element a flattened planar view of a stent as described herein.

FIG. 11C displays a connected view of insertable attachment feature 1110 and receiving attachment feature 1120 of the locking mechanism, comprising upper hook 1113, and lower hook 1114. The hooks can engage into the guiding slots via the translation of the proximal end of the delivery catheter 1050 holding proximal insertable attachment feature 1110 towards the distal end of the delivery catheter 1050 and the receiving attachment feature 1120. Upon engagement, a lock position can be reached that can anchor the retaining latches 1123 and 1121. Upon engagement, the hook and some parts of the insertable attachment feature 1110 can bend or deflect away from the rest of the stent. The bend can be at least 10 degrees, at least 30 degrees, at least 50 degrees, at least 70 degrees, at least 90 degrees, at least 110 degrees, at least 130 degrees, at least 150 degrees, at least 170 degrees, at least 190 degrees, at least 210 degrees, at least 230 degrees, at least 250 degrees, or at least 270 degrees. In some cases, the bend can be less than 10 degrees, less than 30 degrees, less than 50 degrees, less than 70 degrees, less than 90 degrees, less than 110 degrees, less than 130 degrees, less than 150 degrees, less than 170 degrees, less than 190 degrees, less than 210 degrees, less than 230 degrees, less than 250 degrees, or less than 270 degrees. In some cases, the bend can be 180 degrees. FIG. 11D depicts an example embodiment of a locked three-dimensional configuration of an example stent as described herein 1140.

FIG. 12 depicts another example embodiment of a two-dimensional, planar version of a stent 1200 as described herein with an alternate locking mechanism. In some cases, the locking mechanism used with stent 1200 can be an angulated locking mechanism as described below. This closing mechanism can be used with one or more of the distal and proximal locking elements described herein.

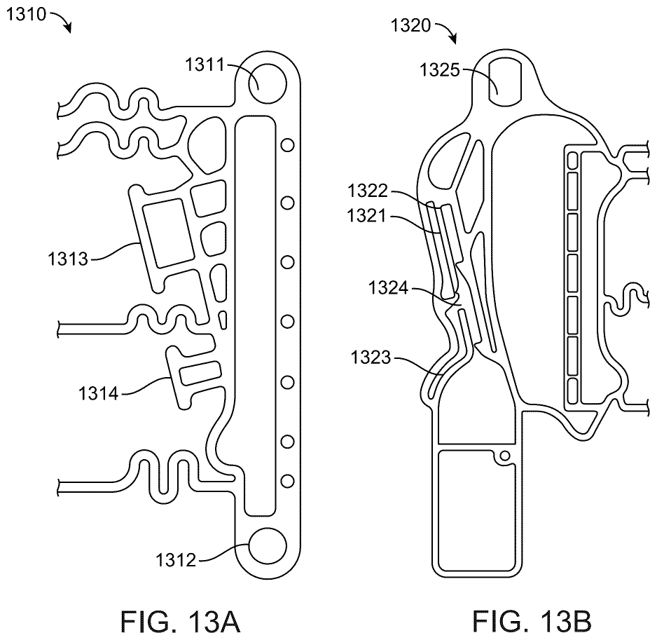

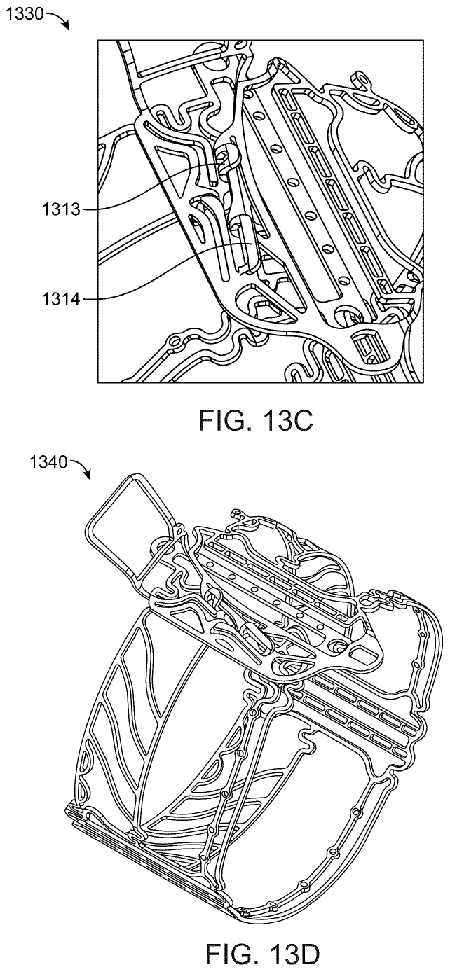

FIG. 13A displays a magnified version of the alternate locking mechanism's insertable attachment feature 1310. Insertable attachment feature 1310 comprises upper proximal attachment feature 1311, lower proximal attachment feature 1312, upper hook 1313, and lower hook 1313. The upper proximal attachment feature 1311 and lower proximal attachment feature 1312 are the most proximal elements of a flattened planar view of a stent as described herein. Upper hook 1313 and lower hook 1314 can be inserted into the receiving attachment feature 1320 upon reaching them through the translation facilitated by the delivery catheter 1050 as described above.

In some cases, insertable attachment feature 1310 can comprise an angulated axial locking mechanism. The translation and locking mechanism can be similar to that discussed above. In some embodiments, upper proximal attachment feature 1311 and lower proximal attachment feature 1312 are not parallel to upper hook 1313 and lower hook 1313. In some cases, upper proximal attachment feature 1311 and lower proximal attachment feature 1312 are angled relative to upper hook 1313 and lower hook 1313. In some cases, this angle can comprise less than 10 degrees, less than 30 degrees, less than 50 degrees, less than 70 degrees, or less than about 90 degrees. In some cases, this angle can comprise greater than 0 degrees, greater than 10 degrees, greater than 30 degrees, greater than 50 degrees, greater than 70 degrees, or greater than about 90 degrees.

FIG. 13B displays a magnified version of the alternate locking mechanism's receiving attachment feature 1320. Receiving attachment feature 1320 of the locking mechanism comprises upper retaining latch 1321, upper cantilevered guiding slot 1322, lower retaining latch 1323, lower cantilevered guiding slot 1324, and upper distal attachment feature 1325. Receiving attachment feature 1320 is the most distal element a flattened planar view of a stent as described herein.

In some cases, receiving attachment feature 1320 can comprise an angulated axial locking mechanism. The translation and locking mechanism can be similar to that discussed above. In some embodiments, receiving attachment feature 1320 is not parallel to the planar axis of the stent. In some cases, receiving attachment feature 1320 is angled relative to the planar axis of the stent. In some cases, this angle can comprise less than 10 degrees, less than 30 degrees, less than 50 degrees, less than 70 degrees, or less than about 90 degrees. In some cases, this angle can comprise greater than 0 degrees, greater than 10 degrees, greater than 30 degrees, greater than 50 degrees, greater than 70 degrees, or greater than about 90 degrees.

In some cases, one or more of upper retaining latch 1321 or lower retaining latch 1323 can be curved. In some embodiments, one of upper retaining latch 1321 and lower retaining latch 1323 is curved while the other is straight. In some cases, lower retaining latch 1323 is curved while upper retaining latch 1321 is straight. In some cases, the angle of curve can comprise less than 10 degrees, less than 30 degrees, less than 50 degrees, less than 70 degrees, or less than about 90 degrees. In some cases, the angle of curve can comprise greater than 0 degrees, greater than 10 degrees, greater than 30 degrees, greater than 50 degrees, greater than 70 degrees, or greater than about 90 degrees.

FIG. 13C displays a connected view of insertable attachment feature 1310 and receiving attachment feature 1320 of the locking mechanism, comprising upper hook 1313, and lower hook 1314. The hooks can engage into the guiding slots via the translation of the proximal end of the delivery catheter 1050 holding proximal insertable attachment feature 1310 towards the distal end of the delivery catheter 1050 and the receiving attachment feature 1320. Upon engagement, a lock position can be reached that can anchor the retaining latches 1323 and 1321. Upon engagement, the hook and some parts of the insertable attachment feature 1310 can bend or deflect away from the rest of the stent. The bend can be at least 10 degrees, at least 30 degrees, at least 50 degrees, at least 70 degrees, at least 90 degrees, at least 110 degrees, at least 130 degrees, at least 150 degrees, at least 170 degrees, at least 190 degrees, at least 210 degrees, at least 230 degrees, at least 250 degrees, or at least 270 degrees. In some cases, the bend can be less than 10 degrees, less than 30 degrees, less than 50 degrees, less than 70 degrees, less than 90 degrees, less than 110 degrees, less than 130 degrees, less than 150 degrees, less than 170 degrees, less than 190 degrees, less than 210 degrees, less than 230 degrees, less than 250 degrees, or less than 270 degrees. In some cases, the bend can be 180 degrees. FIG. 13D depicts an example embodiment of an angulated axial locked three-dimensional configuration of an example deformable stent as described herein.

In some cases, an alternate locking mechanism can be used. In some cases, the alternate locking mechanism can be a radial locking mechanism. A radial locking mechanism is similar to an axial mechanism. The proximal section of the delivery catheter 1050 holding the proximal end of a stent 1400 with a proximal locking element can slide or translate towards the fixed end of the delivery catheter 1050 holding the distal end of a stent 1400 with distal locking element. This mechanism can be used to turn a planar flattened configuration of a stent into a cylindrical or ring structure. The cylinder can be stiff. The stiffness and locking of the stent 1400 can be used to maintain a permanent cylindrical structure of the stent 1400 once deployed into the heart.

FIG. 14A depicts a loading step of a radial closing mechanism comprising delivery catheter 1050 and a stent 1400 as described herein. The lower curving arrow depicts a direction of counterclockwise rotation of mobile proximal portion 1051 prior to loading a stent 1400 onto delivery catheter 1050. The arrow pointing downwards conveys translation toward the proximal end of the delivery catheter 1050 during loading. This closing mechanism can be used with one or more of the distal or proximal locking elements described herein.

FIG. 14B displays a magnified version of the radial locking mechanism's insertable attachment feature 1410. Insertable attachment feature 1410 comprises upper proximal attachment feature 1411, lower proximal attachment feature 1412, upper hook 1413, and lower hook 1414.

FIG. 14C displays a magnified version of the radial locking mechanism's receiving attachment feature 1420. Receiving attachment feature 1420 of the locking mechanism comprises upper cantilevered guiding slot 1422, lower cantilevered guiding slot 1424, and upper distal attachment feature 1425.

The radial locking mechanism can comprise rotating the proximal section of the delivery catheter 1050 counterclockwise. In some cases, the delivery catheter rotates less than about 60 degrees, less than about 120 degrees, less than about 180 degrees, less than about 240 degrees, less than about 300 degrees, or less than about 360 degrees. In some cases, the delivery catheter rotates greater than about 0 degrees, greater than about 60 degrees, greater than about 120 degrees, greater than about 180 degrees, greater than about 240 degrees, or greater than about 300 degrees. In some cases, the delivery catheter rotates 180 degrees. The radial locking mechanism can comprise loading a planar stent 1400 as described herein onto the delivery catheter. While loading the stent 1400 onto the delivery catheter, the radial locking mechanism can comprise translating the proximal section of the delivery catheter 1050 towards the proximal end and away from the fixed distal end of delivery catheter 1050.

After loading is completed, the proximal section of the delivery catheter 1050 holding the proximal end of a stent 1400 with a proximal locking element can slide or translate towards the fixed end of the delivery catheter 1050 holding the distal end of a stent 1400 with distal locking element. FIGS. 14E and 14F depict alternate side views of the translation mechanism to effectuate radial locking of a deformable stent as described herein using delivery catheter 1050. The arrows portray the direction of translation of mobile proximal portion 1051 towards the distal fixed end of the delivery catheter 1050. FIG. 14D depicts an internal view of the delivery catheter 1050 holding stent 1400 pre-translation. FIG. 14G and FIG. 14H display bottom-up and top-down views, respectively. When the delivery catheter 1050 reaches the end of the distal fixed end, the proximal section of delivery catheter 1050 can rotate clockwise. In some cases, the delivery catheter rotates less than about 60 degrees, less than about 120 degrees, less than about 180 degrees, less than about 240 degrees, less than about 300 degrees, or less than about 360 degrees. In some cases, the delivery catheter rotates greater than about 0 degrees, greater than about 60 degrees, greater than about 120 degrees, greater than about 180 degrees, greater than about 240 degrees, or greater than about 300 degrees. In some cases, the delivery catheter rotates 180 degrees. The delivery catheter can rotate clockwise a similar amount as it rotated counterclockwise prior to loading. Rotating clockwise can help the hooks 1413 and 1414 engage into the guiding slots 1422 and 1424 until a lock position is reached. FIG. 14I depicts a side views of the rotation mechanism to effectuate radial locking of a deformable stent as described herein using delivery catheter 1610. The arrow points in the direction of the clockwise rotation to reach the locked position. Once locked, mobile proximal portion 1051 can be released from stent 1400. Guiding slots 1422 and 1424 can comprise a non-return feature that can prevent the proximal end of the stent from translating in the opposite direction away from the distal attachment feature 1420 or slipping out of the slots.

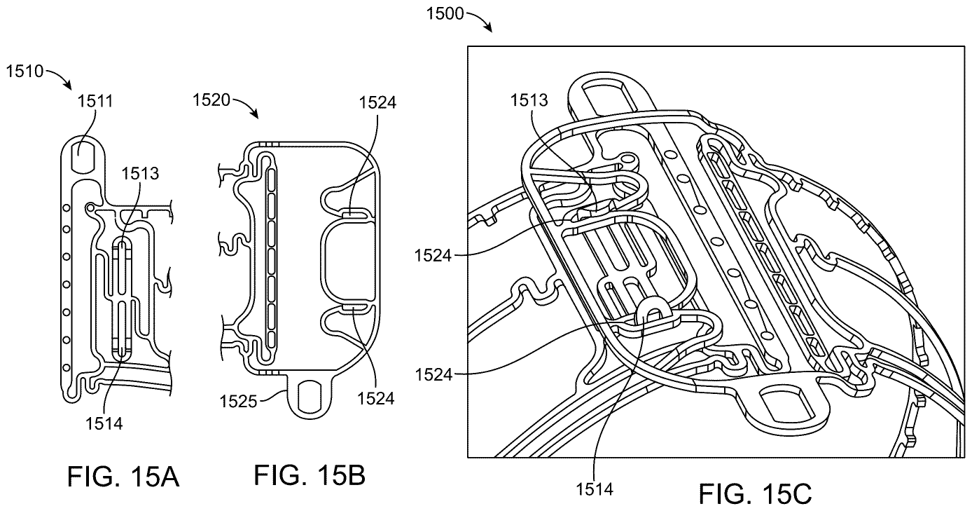

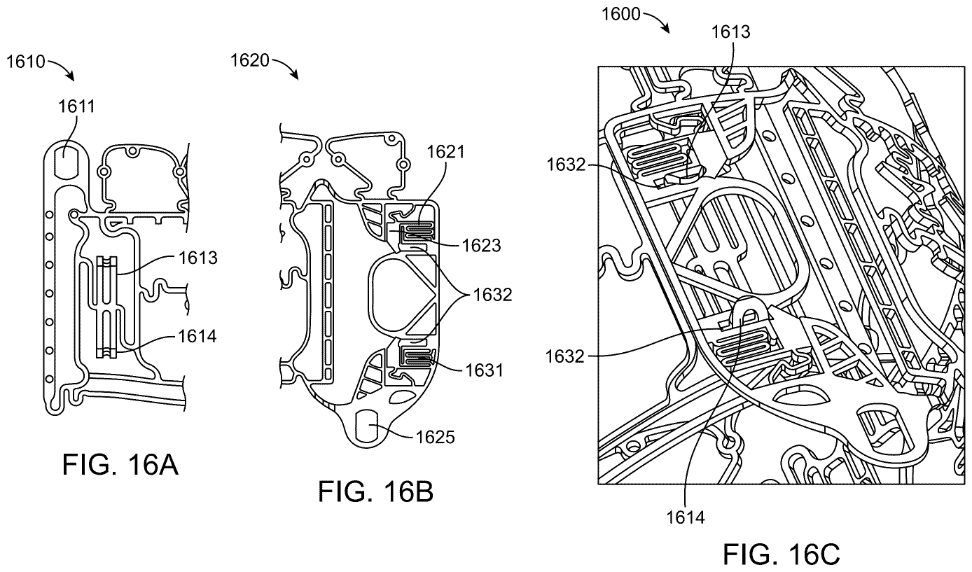

There are many combinations of insertable attachment features and receiving attachment features that can be used with radial locking mechanism, axial locking mechanism, or angulated axial mechanism. FIGS. 15-16 show some additional variations of insertable attachment features and receiving attachment features.

FIG. 15A shows a magnified version of an alternate radial locking mechanism's insertable attachment feature 1510. Insertable attachment feature 1510 comprises upper proximal attachment feature 1511, upper hook 1513, and lower hook 1514, thereby comprising similar features as insertable attachment feature 1410. The functionality can also be similar to insertable attachment feature 1410.

FIG. 15B displays a magnified version of the radial locking mechanism's receiving attachment feature 1520. Receiving attachment feature 1520 of the locking mechanism comprises upper cantilevered guiding slot 1522, lower cantilevered guiding slot 1524, and lower distal attachment feature 1525, thereby comprising similar features as insertable attachment feature 1420. The functionality can also be similar to insertable attachment feature 1420. FIG. 15C depicts an example alternative radial locked three-dimensional configuration of an example deformable stent as described herein.

FIG. 16A shows a magnified version of the alternate radial locking mechanism's insertable attachment feature 1610. Insertable attachment feature 1610 comprises upper proximal attachment feature 2011, upper hook 1613, and lower hook 1614, thereby comprising similar features as insertable attachment feature 1410. The functionality can also be similar to insertable attachment feature 1410.

FIG. 16B displays a magnified version of the radial locking mechanism's receiving attachment feature 1620. Receiving attachment feature 1620 of the locking mechanism can comprise one or more of upper cantilevered guiding slot 1622, lower cantilevered guiding slot 1624, lower distal attachment feature 1625, upper recoil spring 1616, lower recoil spring 1631, upper latch 1623, or lower latch 1632. The functionality can be similar to insertable attachment feature 1420. FIG. 16C depicts an example three-dimensional configuration of an example deformable stent as described herein with the alternate radial locking mechanism of FIGS. 16A and 16B. The latches can provide additional support to maintain the hooks in insertable attachment feature 1610 in receiving attachment feature 1620 by latching onto insertable attachment feature 1610. In some cases, latches 1632 and 1623 can be actuated by an element which acts as a spring. The latches can be pushed when the upper hook 1613 and lower hook 1614 are inserted into the guiding slots 1622 and 1624. The spring can then push the latches 1632 and 1623 to lock the hooks in place. In some cases, the springs are the recoil springs 1631 and 1616.

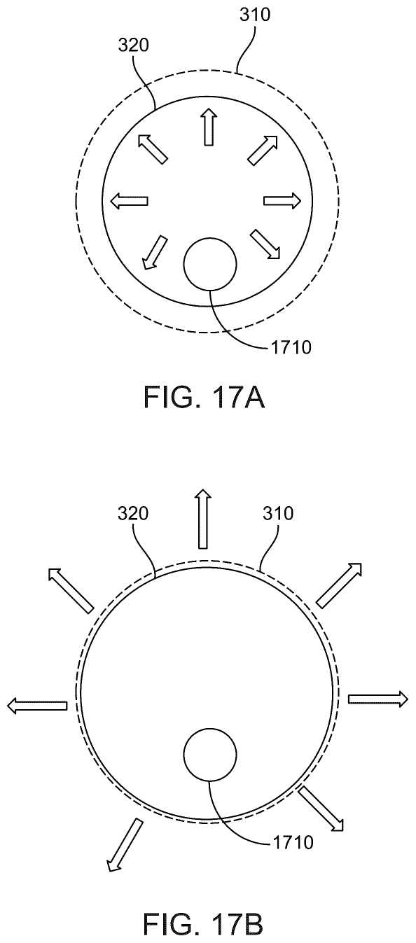

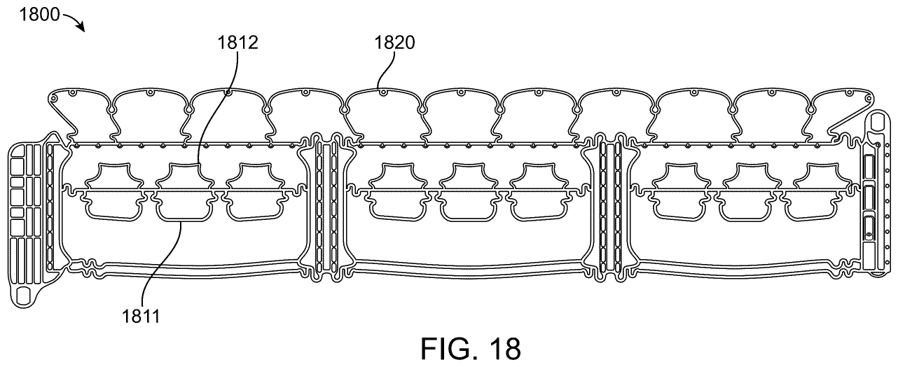

FIGS. 17A-17B provide schematic views of the variable diameter and chronic radial force of the stent. FIG. 17A displays a schematic view of implant 320 onto tissue anatomy 310 and delivery system 1710 before release of the stent. FIG. 17A shows the anatomy 310 in dotted lines and the radial actuation and expansion via the externally pointed arrows. The location of delivery system 1710 is an example location of where the delivery system 1710 can interact with implant 320. FIG. 17B displays a schematic view of implant 320 onto tissue anatomy 310 and delivery system 1710 after release of the stent. In some cases, delivery system 1710 comprises delivery catheter 1050. The implant can comprise any of the embodiments of the deformable stents described herein.

In some embodiments, stent implant 320 is kept circumferentially constrained during the loading phase onto delivery system 1710. In some embodiments, stent implant 320 is kept circumferentially constrained during the deployment phase from delivery system 1710 into the heart valve. The stent structure can then be locked into a cylindrical shape via the locking mechanisms discussed above. When locked into a cylindrical shape, the diameter can originally be smaller than the mitral annulus to be treated. The stent can then be released. The stent diameter can increase on release. The stent diameter can be released by self-actuation. The stent diameter can be released by an external actuation system, for example a balloon catheter. Once released, the stent diameter can be oversized in relation to the native valve anatomy. The oversize can project a chronic radial force onto the native tissue anatomy 310. FIG. 17B displays a view of when the implant has expanded to its full size and is exerting chronic radial force, as portrayed by the externally facing arrows, onto tissue anatomy 310.

The present disclosure can comprise various embodiments of anchoring mechanisms as displayed in FIGS. 18-24. These anchoring mechanisms can be used with any of the stents described herein, alone or in conjunction with any of the features described herein such as, for example, deformation features or locking features. These anchoring mechanisms can provide and maintain a resistance against migration of the stent under the high systolic pressure in the heart during a cardiac cycle. These anchoring mechanisms can provide and maintain a resistance against migration of the stent during the original deployment into the heart.

FIGS. 18-22 can comprise similar elements. These elements can comprise an upper inflow crown, a lower outflow crown, and an intermediate crown. The upper inflow crown can be positioned on the atrium side. It can provide anchoring, stability and migration resistance during deployment of a stent into the heart. The lower outflow crown can be positioned on the ventricular side. It can provide anchoring and migration resistance during high systolic pressures. In some cases, barbs or spikes can be added to the lower outflow crown to increase its ability to anchor the stent. In some cases, the angle of the spikes can be aligned with the one or more of the upper inflow crown or the lower outflow crown. In some cases, the angle of the spikes can be parallel with the heart valve axis. In some cases, the angle of the spikes can be within 45 degrees of the heart valve axis. The angle of the spikes can be within about 10 degrees, 20 degrees, 30 degrees, 40 degrees, or 45 degrees of the heart valve axis. The intermediate crown can be positioned under the mitral annulus. The intermediate crown can be used in conjunction with upper inflow crown to provide anchoring. It can also be used to sustain helicoidal loading of the stent into the heart.

FIG. 18 displays a first variation of an anchoring mechanism 1800 comprising lower outflow crown 1811, intermediate crown 1812, and upper inflow crown 1820. In some cases, the anchoring elements are statically attached to the circumferential rail. The stent's flexibility and accommodation can arise from elements perpendicular to the circumferential rail, for example the commissural stent posts.

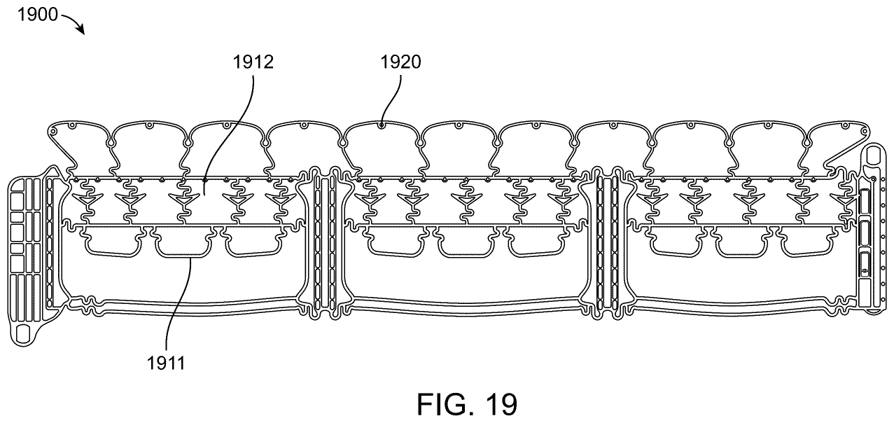

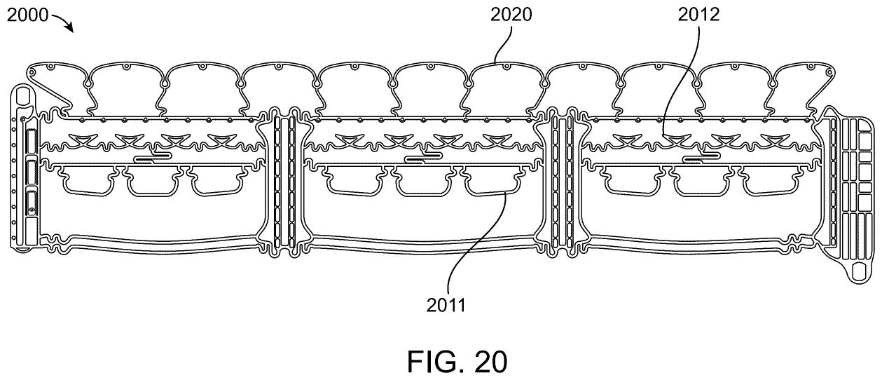

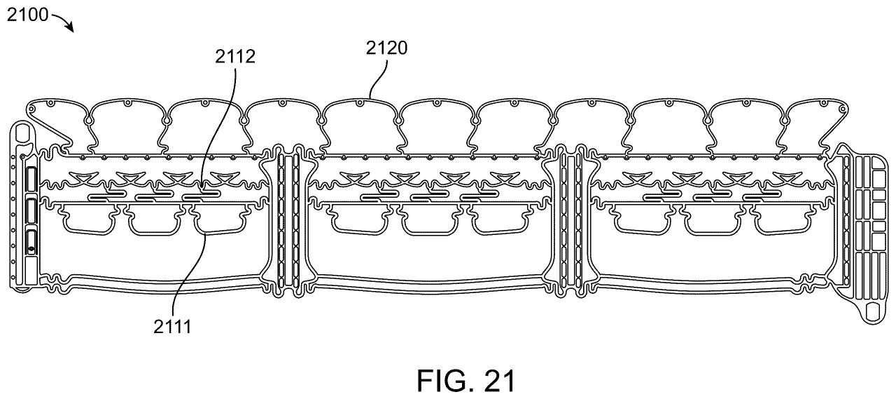

FIG. 19 displays a second variation of an anchoring mechanism 1900 comprising lower outflow crown 1911, intermediate crown 1912, and upper inflow crown 1920. FIG. 20 displays a third variation of an anchoring mechanism 2000 comprising lower outflow crown 2011, intermediate crown 2012, and upper inflow crown 2020. FIG. 21 displays a fourth variation of an anchoring mechanism 2100 comprising lower outflow crown 2111, intermediate crown 2112, and upper inflow crown 2120. FIGS. 19-21 comprise dynamic anchoring elements. These anchoring elements can be dynamic because of the multiple layers of crimping at the circumferential support beams. These dynamic anchoring elements can accommodate and follow annular, leaflet, or ventricular motion in any direction.

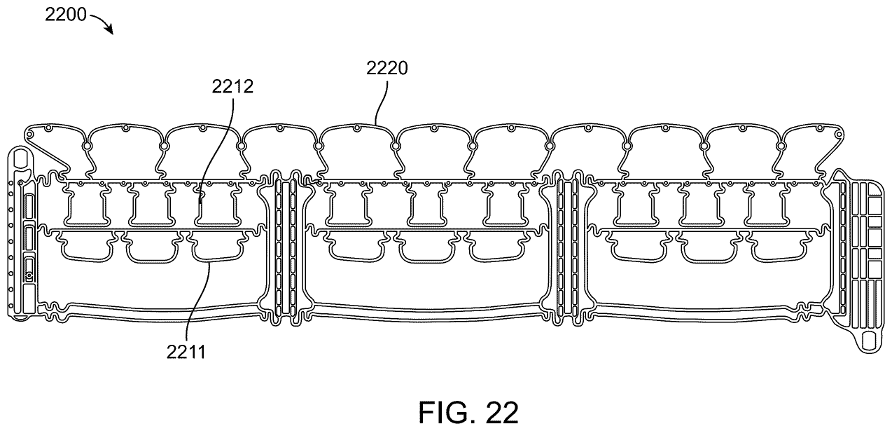

FIG. 22 displays a fifth variation of an anchoring mechanism 2200 comprising lower outflow crown 2211, intermediate crown 2212, and upper inflow crown 2220. The loops in the circumferential support beam can be positioned under the native valves to help anchor the stent.

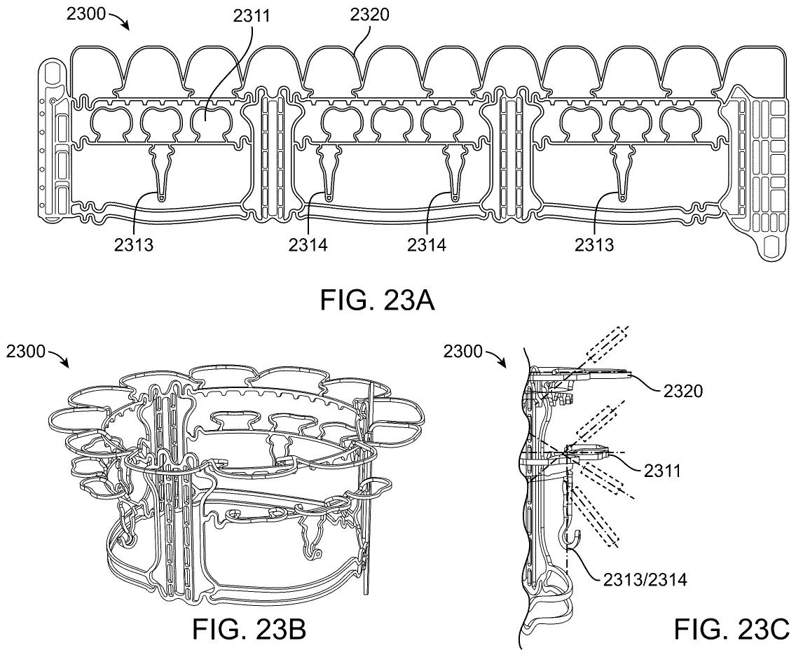

FIG. 23A shows stent an anchoring mechanism 2300 with a ventricular anchoring feature comprising lower outflow crown 2311, posterior native leaflet ventricular anchoring feature 2313, anterior leaflet ventricular anchoring feature 2314, and upper inflow crown 2320. The upper inflow crown 2320 can be positioned on the atrium side. It can provide anchoring, stability and migration resistance during deployment of a stent into the heart. The lower outflow crown 2311 can be positioned on the ventricular side. It can provide anchoring and migration resistance during high systolic pressures. In some cases, barbs or spikes can be added to the lower outflow crown to increase its ability to anchor the stent. Posterior native leaflet ventricular anchoring feature 2313 can comprise an anchoring feature on the ventricular side of the device that can grasp posterior native tissue leaflets. Anterior leaflet ventricular anchoring feature 2314 can comprise an anchoring feature on the ventricular side of the device that can grasp anterior native tissue leaflets. In a planar formation, lower outflow crown 2311, posterior native leaflet ventricular anchoring feature 2313, anterior leaflet ventricular anchoring feature 2314, and upper inflow crown 2320 can all be parallel. In some cases, as shown in FIG. 23C, one or more of the posterior native leaflet ventricular anchoring feature 2313 or anterior leaflet ventricular anchoring feature 2314 can comprise a hook-like design. FIG. 23B shows an anchored three-dimensional configuration of an example deformable stent as described herein.

FIG. 23C displays the variable angles of ventricular anchoring feature 2300 comprising lower outflow crown 2311, posterior native leaflet ventricular anchoring feature 2313, and upper inflow crown 2320. In some cases, the angle of one or more parts of a stent as described herein, for example stent with an anchoring mechanism 2300, can be varied. The angle of one or more parts of a stent as described herein, for example stent with an anchoring mechanism 2300, can be varied by less than about 180 degrees, 160 degrees, 140 degrees, 120 degrees, 100 degrees, 80 degrees, 60 degrees, 40 degrees, or less than about 20 degrees. The angle of one or more parts of a stent as described herein, for example stent with an anchoring mechanism 2300, can be varied by greater than about 160 degrees, 140 degrees, 120 degrees, 100 degrees, 80 degrees, 60 degrees, 40 degrees, 20 degrees or greater than about 0 degrees. In some cases, the angle of two or more parts of a stent as described herein, for example stent with an anchoring mechanism 2300, can be varied by different angles. In some embodiments, the angle of three or more parts can be varied. Upper inflow crown 2320 can be angularly varied. Lower outflow crown 2311 can be angularly varied. Anterior leaflet ventricular anchoring feature 2314 can be angularly varied. Posterior native leaflet ventricular anchoring feature 2313 can be angularly varied.

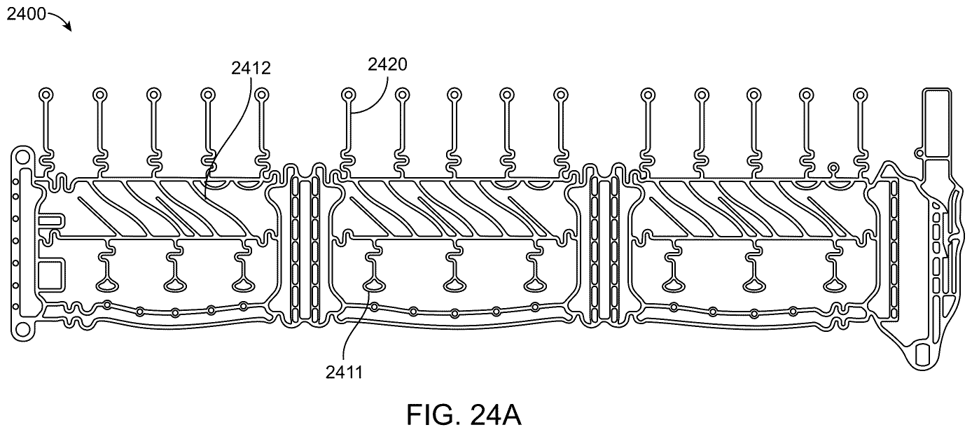

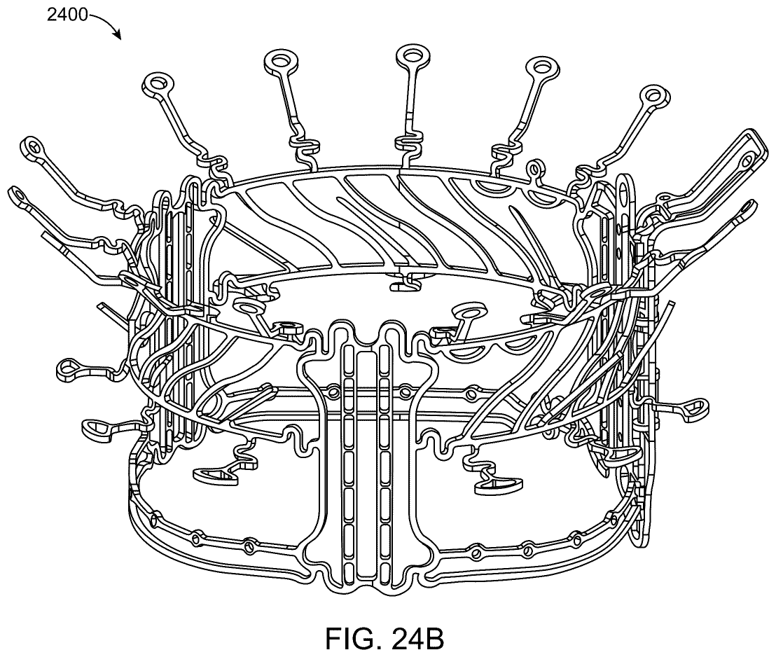

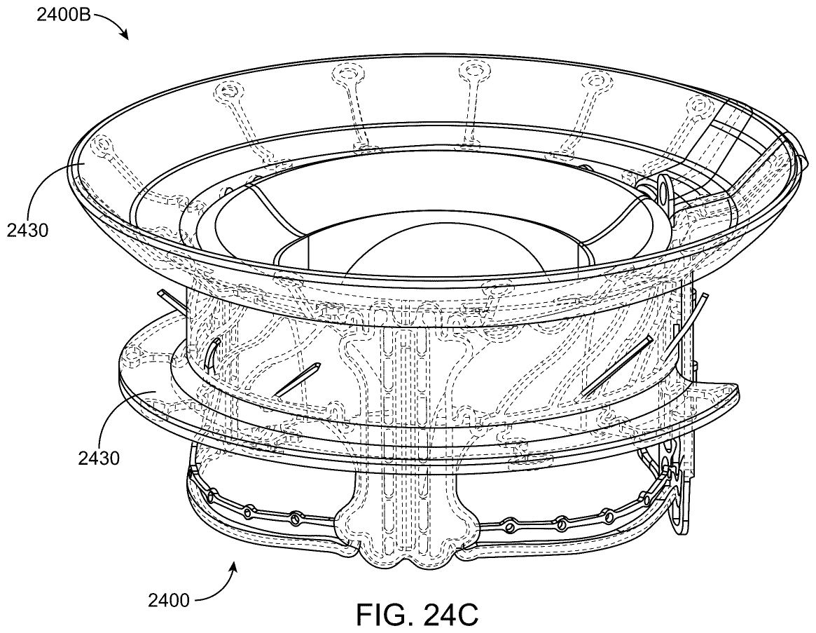

FIGS. 24A-24C show another example of a stent with an anchoring mechanism 2400 with spikes and a covering. FIG. 24A comprises lower outflow crown 2411, upper inflow crown 2420, and annular spikes 2415. The upper inflow crown 2420 can be positioned on the atrium side. It can provide anchoring, stability and migration resistance during deployment of a stent into the heart. Individual crown arms of the upper inflow crown 2420 can be made to conform locally to the tissue anatomy. The lower outflow crown 2411 can be positioned on the ventricular side. It can provide anchoring and migration resistance during high systolic pressures. In some cases, additional barbs or spikes can be added to the lower outflow crown to increase its ability to anchor the stent. Annular spikes 2415 can be positioned between upper inflow crown 2420 and lower outflow crown 2411. Annular spokes 2415 can penetrate tissue anatomy. This spike positioning can help sustain helicoidal loading. FIG. 24B shows an anchored three-dimensional configuration of an example embodiment of a stent as described herein 2400.

FIG. 24C comprises valve prosthesis 2400B comprising covering 2430 surrounding stent 2400. Stent 2400 comprises lower outflow crown 2411, upper inflow crown 2420, and annular spikes 2415 as described above. The covering can link individual arms of the upper inflow crown 2420. The covering can link individual arms of the upper inflow crown 2420 without impeding the ability of each crown arm to conform to tissue anatomy.

### Leaflet Configuration

Provided herein are devices for insertion into a heart of a subject and configured to be coupled to an intraluminal support structure, comprising a scaffold panel having a first free end and a second free end when the scaffold panel is in a flattened configuration. The first and second free ends can be configured to be coupled to each other to form into a ring when deployed, transitioning the valve structure from a flattened configuration to a ring configuration. The valve structure can comprise one or more support bodies configured to form a central channel when the valve structure is in the ring configuration; and one or more central leaflet cusps; and one or more connecting skirts. The one or more central leaflet cusps can be inserted into the arch of the one or more valve support bodies. The one or more connecting skirts are attached to the bottom, or the atrium side, of the valve support bodies. The one or more support bodies, one or more central leaflet cusps, and one or more connecting skirts can make up the biological components of the valve prosthesis and are located internal to the stents described herein when in the deployed configuration. The biological components and the stents can comprise the valve prosthesis.

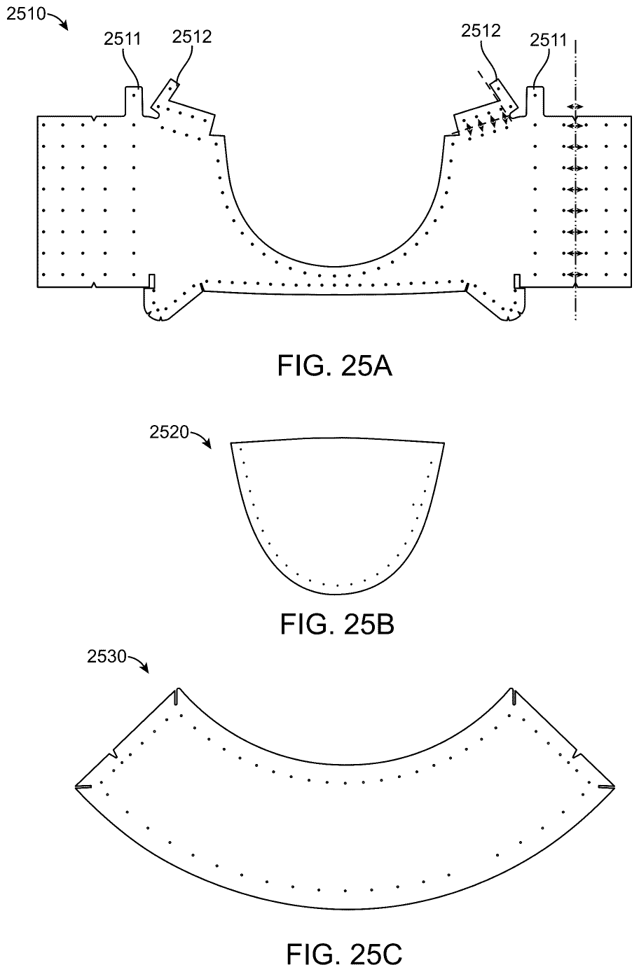

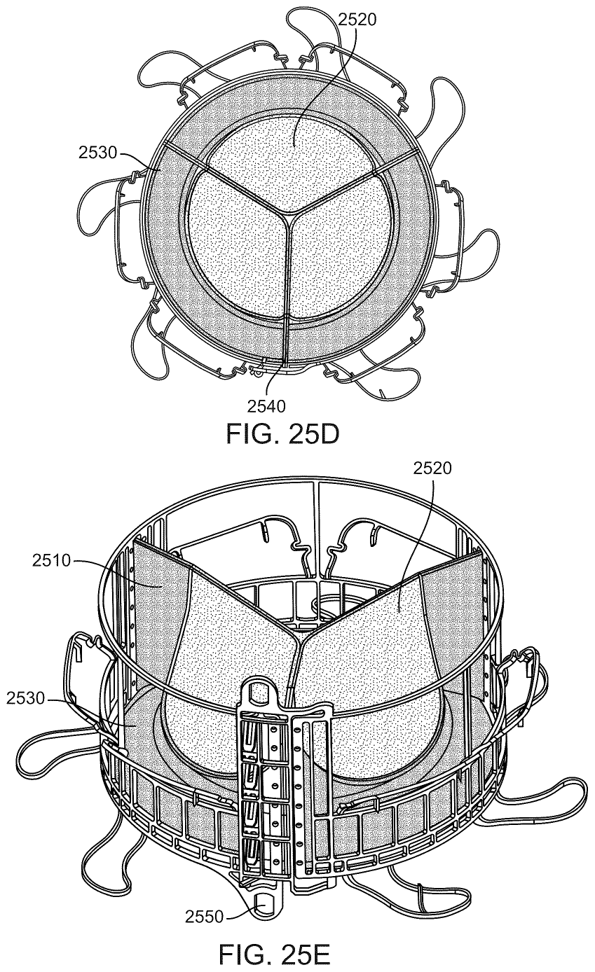

FIGS. 25A-25C depict parts of the valve comprising valve support body 2510, central leaflet cusp 2520, and connecting skirt 2530. Each valve can comprise one or more valve support bodies 2510, central leaflet cusps 2520, and connecting skirts 2530. Each valve can comprise three valve support bodies 2510, central leaflet cusps 2520, and connecting skirts 2530. Each valve can comprise two valve support bodies 2510, central leaflet cusps 2520, and connecting skirts 2530. Each valve can comprise one or more sub-parts, wherein each sub-part can comprise one or more valve support bodies 2510, one or more central leaflet cusps 2520, and one or more connecting skirts 2530. Each sub-part can comprise one valve support body 2510, one central leaflet cusp 2520, and one connecting skirt 2530.

FIG. 25A shows valve support body 2510 comprising reinforcement tabs 2512 and locking tabs 2511. Reinforcement tabs 2512 can help to prevent commissural tearing. Locking tabs 2511 can attach valve support bodies 2510 to one another. Locking tabs 2511 can do so by folding over the tops of neighboring valve support bodies 2510 at the commissure top. The commissure top is the crease between each sub-part of the valve. Valve support body 2510 can comprise various materials, including but not limited to biological tissue, for example biological valve tissue, polymers, or a hybrid tissue composition. The hybrid tissue composition can comprise a mix of biological tissue, for example biological valve tissue and polymers. Polymers can include, but are not limited to, polyurethane elastomers, silicone, and polyethylene terephthalate.

FIG. 25B shows central leaflet cusp 2520. Central leaflet cusp 2520 can be flattened during loading of the valve prosthesis to allow the commissural post to open. Central leaflet cusp 2520 returns to the non-flattened position once the valve prosthesis is deployed. Central leaflet cusp 2520 can comprise various materials, including but not limited to biological tissue, for example biological valve tissue, polymers, or a hybrid tissue composition. The hybrid tissue composition can comprise a mix of biological tissue, for example biological valve tissue and polymers. Polymers can include, but are not limited to, polyurethane elastomers, silicone, and polyethylene terephthalate.

FIG. 25C shows connecting skirt 2530. Connecting skirt 2530 can compensate for a diameter discrepancy between the central orifice of the heart valve into which the stent was deployed and the stent diameter. Connecting skirt 2530 can be attached to the stent structure. In some cases, central leaflet cusps 2520 can have a fixed diameter regardless of the stent structure diameter. To account for this, connecting skirt 2530 can be variable in diameter. Connecting skirt 2530 can dampen central leaflet cusp 2520 during valve operation. Connecting skirt 2530 can comprise various materials, including but not limited to biological tissue, for example biological valve tissue, polymers, or a hybrid tissue composition. The hybrid tissue composition can comprise a mix of biological tissue, for example biological valve tissue and polymers. Polymers can include, but are not limited to, polyurethane elastomers, silicone, and polyethylene terephthalate.

Valve support body 2510, central leaflet cusp 2520, and connecting skirt 2530 can be assembled together to form the biological components of the valve prosthetic. One or more of these parts can be assembled together by sewing, gluing, or other methods of bonding. In some cases, the arched valley of valve support body 2510 is bonded to the arch of central leaflet cusp 2520. In some cases, the feet of valve support body 2510 are bonded to the right and left sides of connecting skirt 2530. The bottom part, or atrium-facing part, of valve support body 2510 between the feet can be bonded to the inner-facing part of connecting skirt 2530.

In some cases, one or more sub-parts of the valve can be preassembled. In some cases, one or more sub-parts can be custom-made for a patient. In some cases, all sub-parts of the valve can be preassembled. In some cases, valve supporting body 2510 can be preassembled. In some cases, central leaflet cusp 2520 can be preassembled. In some embodiments, connecting skirt 2530 can be preassembled. Preassembly can improve uniformity of the sub-parts of the valve. Uniformity can improve matching of sub-parts for better valve hydrodynamics. In some cases, each sub-part can be attached individually to the stent structure. In some embodiments, sub-parts are combined and then attached to the stent structure.

FIG. 25D shows a top-down view of the assembled sub-parts inserted into a stent as described herein, comprising central leaflet cusps 2520, connecting skirts 2530, and commissural stent posts 2540. In some cases, the one or more central leaflet cusps 2520 have a fixed diameter regardless of the stent structure diameter. The connecting skirts 2530 can have a variable diameter to account for the different between the diameter of the central leaflets 2520 and the stent structure. In some cases, the one or more central leaflet cusps 2520 flatten during loading of the valve prosthesis, allowing the commissural posts 2540 to open. Once the commissural posts 2540 close, the one or more central leaflet cusps 2520 return to their 3D configuration as shown in FIG. 25E. In some embodiments, there can be three central leaflet cusps 2520. In some embodiments, there can be two central leaflet cusps 2520.

FIG. 25E shows a perspective view of the assembled sub-parts inserted into a stent as described herein, comprising central leaflet cusps 2520, connecting skirts 2530, valve support bodies 2510, and locking mechanism 2550. Connecting skirt 2530 can be held up by valve support body 2510. The sides of the valve support body 2510 can meet at the commissural stent posts. In some cases, the valve support bodies in each of the one or more sub-parts are not attached to the valve support bodies of the other sub-parts. In some cases, the sides of the valve support body 2510 can create commissural flaps where they do not fully meet the commissural stent posts. The commissural flaps can open during diastole.

### Gap Filling Support Structure

Described herein are gap filling support structures for use with one or more of the stents, locking mechanisms, or leaflet configurations described herein. Described herein are intraluminal support structures for insertion into a heart valve of a subject comprising a scaffold panel. The scaffold panel can have a first free end and a second free end when the scaffold panel is in a flattened configuration. The first and second free ends can be configured to be coupled to each other to form into a ring when deployed. The scaffold panel can comprise a circumferential supporting portion spanning from the first free end to the second free end. The scaffold panel can comprise a plurality of gap filling arms extending from the circumferential supporting portion. In some cases, at least a terminal portion of the plurality of gap filling arms is parallel and offset from the circumferential supporting portion. In some cases, the plurality of gap filling arms can define a cavity with a round or D-shaped cross-section around the circumferential supporting portion. In some cases, the plurality of gap filling arms fills a void between the circumferential supporting portion and an anatomy of the heart valve. The plurality of gap filling arms can create a paravalvular leak sealing volume. The plurality of gap filling arms can dampen the force of the heart valve on the intraluminal support structure, allowing the intraluminal support structure to maintain a cylindrical valve orifice. In some cases, the intraluminal support structure is non-deformable. In some cases, the intraluminal support structure is deformable. In some embodiments, the intraluminal support structure is larger than the heart valve in an anterior-posterior direction and exerts force on the heart valve, thereby pushing the gap filling arms outward.

In some cases, the gap filling structure can comprise nitinol. The gap filling structure can comprise a nitinol tube. The gap filling structure can comprise a laser cut nitinol tube. In some cases, the gap filling structure is self-expandable to fill gaps. In some cases, the gap filling structure is manually expandable to fill gaps. The gap filling structure can comprise other materials, including but not limited to stainless steels, other metallic alloys, nylon polymers, polyurethane, and other polymers.

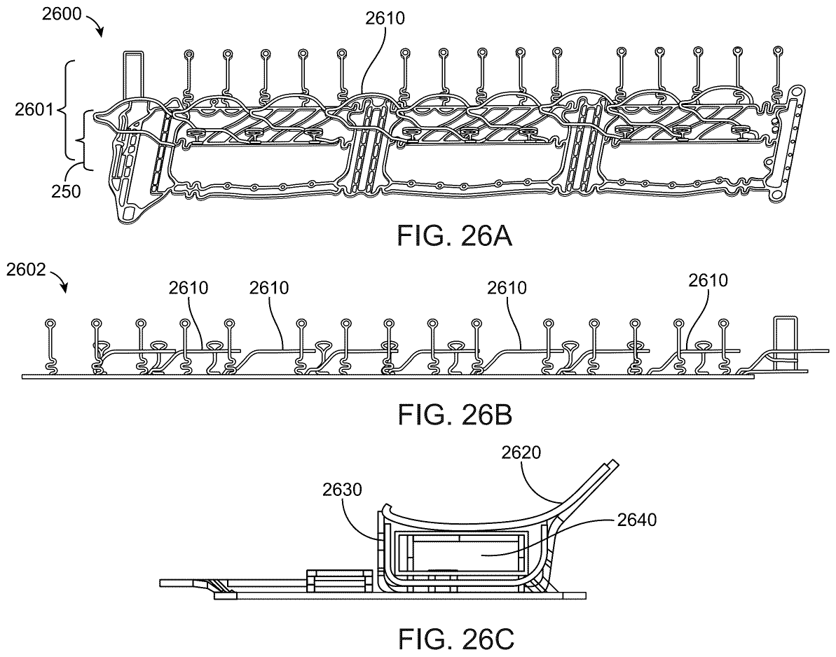

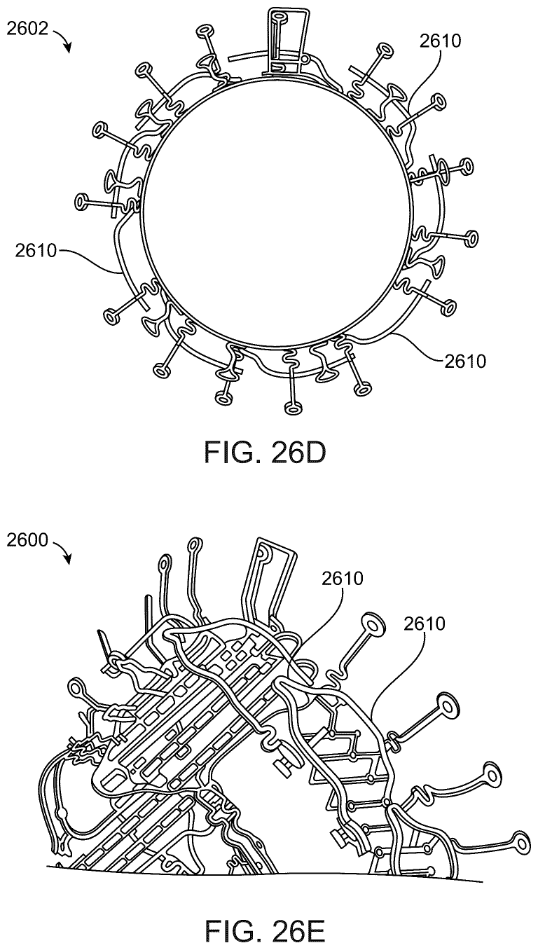

FIGS. 26A-26E show an example of a stent 2600 with a gap filling supporting structure 2601. FIG. 26A displays gap filling supporting structure 2601 comprising gap filling arms 2610 and circumferential supporting beam 2620. Circumferential supporting beam 2620 can be analogous to circumferential supporting beam 250 and other circumferential supporting beams described herein. FIG. 26B displays gap filling arms 2610 interspersed among the upper inflow crown 2602 of an example stent 2600. FIG. 26C depicts a side view of gap filling supporting structure 2601 comprising cavity 2640, inner covering 2630, and outer covering 2620. Gap filling arms 2610 can define a cavity 2640 around the circumferential supporting beam 2620. The cavity 2640 can create a paravalvular leak sealing volume. FIGS. 26D and 26E display additional views of an example stent with a gap filling supporting structure 2600. FIG. 26D shows a top-down view comprising gap filling arms 2610 interspersed among the upper inflow crown 2602 of an example stent. FIG. 26E shows a side view of an example stent 2600 comprising gap filling arms 2610. Gap filling arms 2610 can be used with any stent as described herein.

A gap filling support structure can fill a void between a circumferential supporting beam, for example circumferential supporting beam 2620, and the native tissue anatomy. In some cases, the void can be variable, and the gap filling support structure can fill the variable void. The diameter of the gap filling arms 2610 can be larger than the diameter of the circumferential support beam 2620. For this reason, the gap filling supporting structure 2601 can be used to define a stent diameter and can be used to derive the degree of oversize, wherein the degree of oversize is relevant as described above. In some cases, gap filling can be used to conform a stent as described herein to unevenness of native heart tissue anatomy. Gap filling can also be used to reinforce a stent's resistance against migration due to systolic forces. The gap filling arms 2610 can provide a supporting structure for an outer covering of a stent.

In some cases, gap filling arms 2610 can be used with a non-deformable stent as described above. In some embodiments, a gap filling support structure 2601 can be used with a deformable stent as described above.

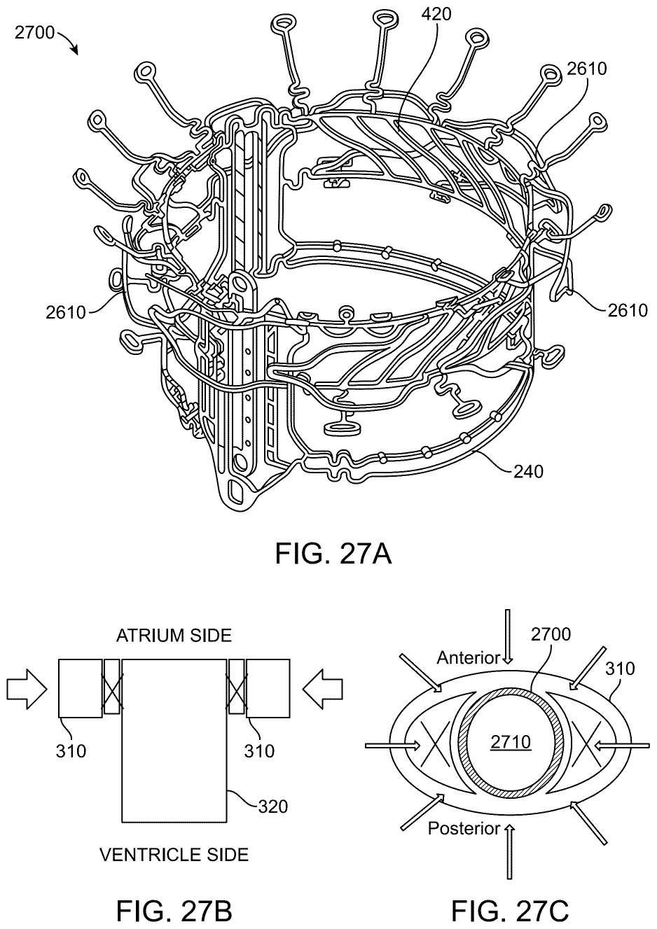

FIGS. 27A-27C display a first configuration of stent 2700 with a gap filling structure with a non-deformable circumferential support beam. FIG. 27A displays non-deformable stent 2700 comprising gap filling arms 2610, circumferential rail 240, and non-deformable circumferential support beam 420. In this configuration, the stent size matches the anterior-posterior dimension of the native valve annulus.

Non-deformable circumferential support beam 420 is similar to circumferential support beam 250. Non-deformable circumferential support beam 420 can be parallel to the upper inflow crown. Non-deformable circumferential support beam 420 can be parallel to the circumferential rail. Non-deformable circumferential support beam 420 can be perpendicular to the commissural stent post. Non-deformable circumferential support beam 420 can be perpendicular to the proximal locking mechanism. Non-deformable circumferential support beam 420 can be perpendicular to the distal locking mechanism.

Non-deformable circumferential support beam 420 can provide circumferential stiffness to a stent as described herein. Non-deformable circumferential support beam 420 can facilitate low radial deformation of a stent. Non-deformable circumferential support beam 420 can help an inserted stent resist crushing forces of native tissue of the heart. Non-deformable circumferential support beam 420 can define a sealing zone around the mitral annulus of the heart. Non-deformable circumferential support beam 420 can support a sealing skirt of a stent as described herein by facilitating attachment of a valve skirt by protecting the skirt from migration and movement due to blood flow.

In some embodiments, the circumferential rails used in stent 2700 are circumferential rails 240. In some embodiments, the circumferential rails used in stent 2700 are other circumferential rails described herein. Circumferential rails follow the circumference of a stent. In some cases, there is a single circumferential rail following the entire circumference. In some embodiments, there are multiple sections of circumferential rails separated by commissural stent posts. In some embodiments, there are three circumferential rail sections separated by commissural stent posts. In some cases, each section of circumferential rail can comprise one or more struts. In some cases, each section of circumferential rail comprises two struts. Circumferential rails can be the part of the stent furthest from an upper inflow crown. Circumferential rails can be parallel to the upper inflow crown. Circumferential rails can be parallel to a lower outflow crown. Circumferential rails can be parallel to a circumferential supporting beam. Circumferential rails can be perpendicular to commissural stent posts. Circumferential rails can be perpendicular to a proximal locking mechanism. Circumferential rails can be perpendicular to a distal locking mechanism. In some cases, circumferential rails can limit commissural tip displacement. In some cases, circumferential rails can prevent interaction between native tissue leaflets and stent valve leaflets by establishing a perimeter of a stent. In some embodiments, circumferential rails can comprise rail strain relief feature. Rail strain relief features can be a small crimp near the edges of the circumferential rail where it meets commissural stent posts.

A gap filling support structure can fill a void between a circumferential supporting beam, for example circumferential supporting beam 2620, and the native tissue anatomy. In some cases, the void can be variable, and the gap filling support structure can fill the variable void. The diameter of the gap filling arms 2610 can be larger than the diameter of the circumferential support beam 2620. For this reason, the gap filling supporting structure 2601 can be used to define a stent diameter and can be used to derive the degree of oversize, wherein the degree of oversize is relevant as described above. In some cases, gap filling can be used to conform a stent as described herein to unevenness of native heart tissue anatomy. Gap filling can also be used to reinforce a stent's resistance against migration due to systolic forces. The gap filling arms 2610 can provide a supporting structure for an outer covering of a stent. Gap filling arms 2610 can act as a damping member for the acting force of tissue anatomy 310 onto stent 2700. The flexibility of the gap filling arms 2610 and gap filling supporting structure can help a stent conform to the surrounding anatomy.

FIG. 27B shows a schematic view of the interactions between tissue anatomy 310 and implant 320 on the atrial-ventricular axis. The arrows portray the acting force from the tissue anatomy 310 onto the implant 320, wherein implant 320 can comprise non-deformable stent 2700. The “Xs” indicate the space taken up by gap filling arms 2610, indicating the damping effort of gap filling arms 2610 as described above. FIG. 27C shows a schematic view of forces, comprising tissue anatomy 310, on a non-deformable stent with a gap filling structure 2700 as described herein and valve orifice 2710 on the anterior-posterior axis. The arrows portray the acting force from the tissue anatomy 310 onto the non-deformable stent 2700. Valve orifice 2710 of stent 2700 can remain cylindrical once implanted. Remaining cylindrical can improve stent function and durability.

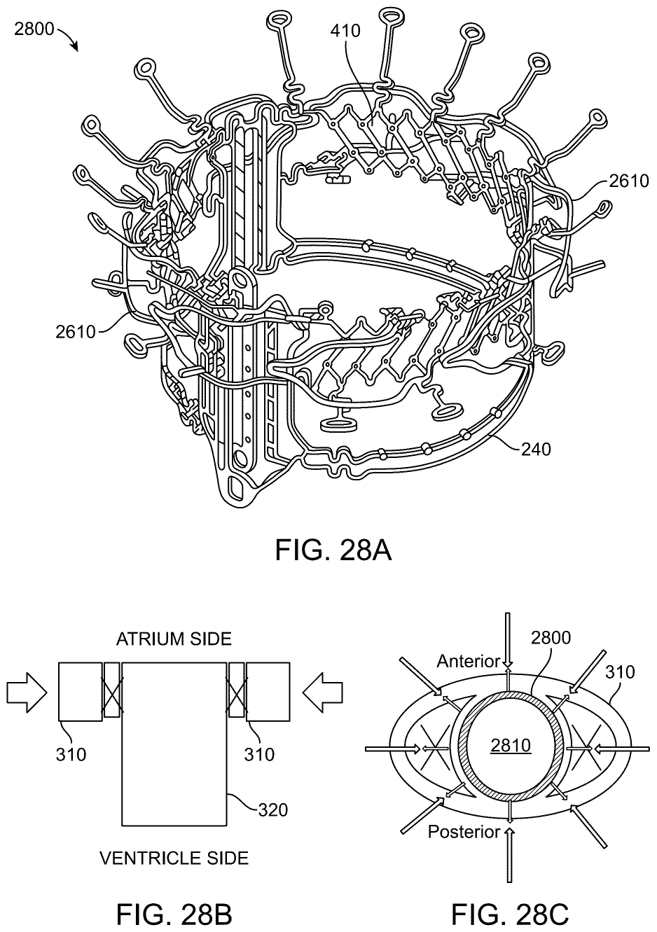

FIGS. 28A-28C depict a second configuration of stent 2800 with a gap filling structure with a deformable circumferential support beam. FIG. 28A displays second configuration of a gap filling structure 2800 comprising gap filling arms 2610, circumferential rail 240, and deformable circumferential support beam 410. In some cases, stent 2800 can be larger than the anterior-posterior dimension of the native valve annulus. This can create a chronic radial force on the surrounding native tissue. This can push the gap filling structure outward. The flexibility of the gap filling arms 2610 and gap filling supporting structure can help a stent conform to the surrounding anatomy.

A gap filling support structure can fill a void between a circumferential supporting beam, for example circumferential supporting beam 2620, and the native tissue anatomy. In some cases, the void can be variable, and the gap filling support structure can fill the variable void. The diameter of the gap filling arms 2610 can be larger than the diameter of the circumferential support beam 2620. For this reason, the gap filling supporting structure 2601 can be used to define a stent diameter and can be used to derive the degree of oversize, wherein the degree of oversize is relevant as described above. In some cases, gap filling can be used to conform a stent as described herein to unevenness of native heart tissue anatomy. Gap filling can also be used to reinforce a stent's resistance against migration due to systolic forces. The gap filling arms 2610 can provide a supporting structure for an outer covering of a stent.

In some embodiments, the circumferential rails used in stent 2800 are circumferential rails 240. In some embodiments, the circumferential rails used in stent 2800 are other circumferential rails described herein. Circumferential rails follow the circumference of a stent. In some cases, there is a single circumferential rail following the entire circumference. In some embodiments, there are multiple sections of circumferential rails separated by commissural stent posts. In some embodiments, there are three circumferential rail sections separated by commissural stent posts. In some cases, each section of circumferential rail can comprise one or more struts. In some cases, each section of circumferential rail comprises two struts. Circumferential rails can be the part of the stent furthest from an upper inflow crown. Circumferential rails can be parallel to the upper inflow crown. Circumferential rails can be parallel to a lower outflow crown. Circumferential rails can be parallel to a circumferential supporting beam. Circumferential rails can be perpendicular to commissural stent posts. Circumferential rails can be perpendicular to a proximal locking mechanism. Circumferential rails can be perpendicular to a distal locking mechanism. In some cases, circumferential rails can limit commissural tip displacement. In some cases, circumferential rails can prevent interaction between native tissue leaflets and stent valve leaflets by establishing a perimeter of a stent. In some embodiments, circumferential rails can comprise rail strain relief feature. Rail strain relief features can be a small crimp near the edges of the circumferential rail where it meets commissural stent posts.

Deformable circumferential support beam 410 can comprise quadrilaterals comprised of strut separators. Deformable circumferential support beam 410 can comprise rectangles, parallelograms, squares, rhombi, trapezoids, kites, or any other type of quadrilateral comprised of strut separators. In some embodiments, the areas where the strut separators meet form V-shaped cells. In some cases, deformable circumferential support beams do not comprise upper and lower circumferential struts. The long edges of the quadrilaterals comprise stiff connecting members. Quadrilateral strut separators can compress like an accordion at their V-shaped cells, giving them the ability to deform without permanently altering the shape of the stent. This can provide a stent structure with a variable perimeter length. The quadrilateral strut separators can comprise a propensity to return to their original perimeter, thus providing a chronic outward radial force while deformed.

In some cases, the quadrilateral strut separators can compress to less than 10%, less than 20%, less than 30%, less than 40%, less than 50%, less than 60%, less than 70%, less than 80%, less than 90%, or less than 100% of their full length.

Deformable circumferential support beam 410 can define a sealing zone around the mitral annulus. Deformable circumferential support beam 410 can support a sealing skirt of a stent as described herein by facilitating attachment of a valve skirt by protecting the skirt from migration and movement due to blood flow. The functionality of deformable circumferential support beam 410 can be similar to circumferential support beam 250.

FIG. 28B shows a schematic view of the interactions between tissue anatomy 310 and implant 320 on the atrial-ventricular axis. The arrows portray the acting force from the tissue anatomy 310 onto the implant 320, wherein implant 320 can comprise deformable stent 2800. Gap filling arms 2610 can act as a damping member for the acting force of tissue anatomy 310 onto stent 2800. The “Xs” indicate the space taken up by gap filling arms 2610, indicating the damping effort of gap filling arms 2610. FIG. 28C shows a schematic view of forces, comprising tissue anatomy 310, on a deformable stent with gap filling structure 2800 as described herein and valve orifice 2810 on the anterior-posterior axis. Valve orifice 2810 of stent 2800 can remain cylindrical once implanted. Remaining cylindrical can improve stent function and durability. The inward arrows portray the acting force from the tissue anatomy 310 onto the non-deformable stent 2800. The two outward arrows portray the chronic radial force pushing the gap filling supporting structure and the stent outwards due to the stent's larger size.

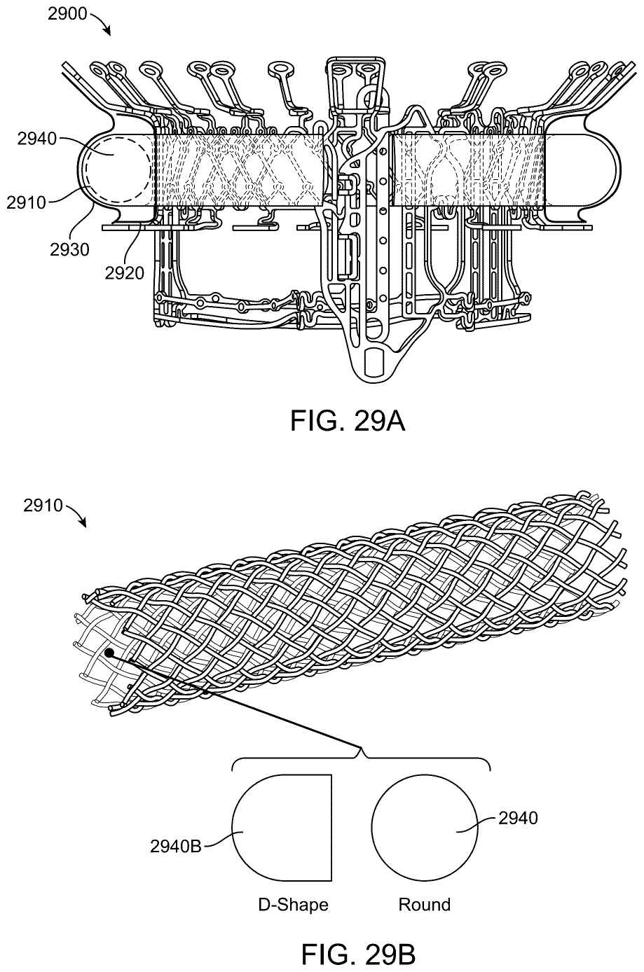

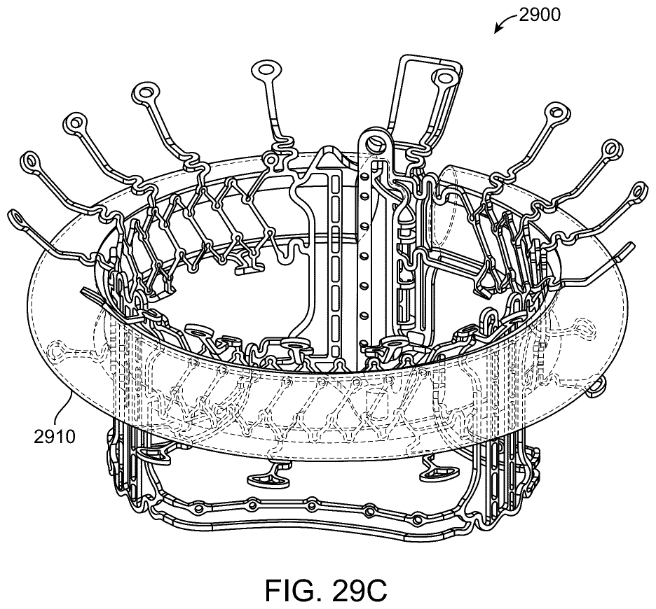

FIGS. 29A-29C depict a gap filling supporting structure surrounding a stent 2900 as described herein. FIG. 29A shows a side view of the insides of a stent with a gap filling supporting structure 2900 as described herein comprising outer covering 2930, inner covering 2920, cavity 2940, and gap filling supporting structure 2910. Outer covering 2930, inner covering 2920, and cavity 2940 can be similar in functionality and appearance to outer covering 2620, inner covering 2630, and cavity 2640, respectively. Gap filling supporting structure 2910 can be similar in functionality and appearance to gap filling supporting structure 2610. Gap filling supporting structure 2910 can be similar in functionality and different in appearance to gap filling supporting structure 2610.

In some cases, the intraluminal support structure can comprise gap filling arms with an open toroidal braided structure comprising nitinol or polymeric material. Gap filling supporting structure 2910 can comprise an open toroidal braided structure. The braided structure can comprise nitinol or polymeric material, for example braided nitinol. The braided structure can comprise other materials, including but not limited to stainless steels, other metallic alloys, nylon polymers, polyurethane, and other polymers. In some cases, gap filling structure 2910 is not a braided structure. The gap filling structure 2910 can comprise a laser cut nitinol tube. In some embodiments gap filling structure 2910 is not toroidal or rounded. In some cases, the gap filling structure is self-expandable to fill gaps. In some cases, the gap filling structure is manually expandable to fill gaps.

FIG. 29B displays a magnified version of the cross-sections of gap filling supporting structure 2910 comprising round cavity 2940 or D-shaped cavity 2940B. In some cases, gap filling structure 2910 can comprise a rounded cavity cross-section 2940. In some embodiments, gap filling structure 2910 can comprise a D-shaped cavity cross-section 2940B. The functionality of cavity 2940 and 2940B can be similar. In some cases, cross-sections gap filling structure can be other shapes, including but not limited to rectangles, parallelograms, squares, rhombi, trapezoids, kites, any other type of quadrilateral, ovals, other types of ellipses, or triangular. FIG. 29C displays an expanded out view of gap filling supporting structure in a stent as described herein 2900.

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity or for ready reference, and the inclusion of such definitions herein should not necessarily be construed to represent a substantial difference over what is generally understood in the art.

The word “stent” can be understood to be synonymous with “intraluminal support structure.” The stent, or intraluminal support structure, can provide the skeleton or frame of a heart valve prosthesis.

The words “upper inflow crown” can be understood to be synonymous with “superior inflow crown” and with “atrial inflow crown.” The words “lower outflow crown” can be understood to be synonymous with “inferior outflow crown” and with “ventricular outflow crown.”

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The ranges disclosed herein also encompass any and all overlap, sub-ranges, and combinations thereof. Language such as “up to,” “at least,” “greater than,” “less than,” “between,” and the like includes the number recited. Numbers preceded by a term such as “approximately”, “about”, and “substantially” as used herein include the recited numbers, and also represent an amount close to the stated amount that still performs a desired function or achieves a desired result. The term “about” or “approximately” may mean within an acceptable error range for the particular value, which will depend in part on how the value is measured or determined, e.g., the limitations of the measurement system. For example, the terms “approximately”, “about”, and “substantially” may refer to an amount that is within less than 10% of, within less than 5% of, within less than 1% of, within less than 0.1% of, and within less than 0.01% of the stated amount. For example, “about” may mean within 1 or more than 1 standard deviation, per the practice in the art. Alternatively, “about” may mean a range of up to 20%, up to 10%, up to 5%, or up to 1% of a given value. As used herein, the term “about” a number refers to that number plus or minus 10% of that number. The term “about” a range refers to that range minus 10% of its lowest value and plus 10% of its greatest value. Where particular values are described in the application and claims, unless otherwise stated the term “about” meaning within an acceptable error range for the particular value may be assumed.

As used in the specification and claims, the singular forms “a”, “an” and “the” include plural references unless the context clearly dictates otherwise. For example, the term “a sample” includes a plurality of samples, including mixtures thereof.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

The terms “subject,” “individual,” or “patient” are often used interchangeably herein. A “subject” can be a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject may be diagnosed or suspected of being at high risk for a disease. In some cases, the subject is not necessarily diagnosed or suspected of being at high risk for the disease.

As used herein, the term “about” a number refers to that number plus or minus 10% of that number. The term “about” a range refers to that range minus 10% of its lowest value and plus 10% of its greatest value.

As used herein, the terms “treatment” or “treating” are used in reference to a pharmaceutical or other intervention regimen for obtaining beneficial or desired results in the recipient. Beneficial or desired results include but are not limited to a therapeutic benefit or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

The section headings used herein are for organizational purposes only and are not to be construed as limiting the subject matter described.

While embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1.-14. (canceled)

15. An intraluminal support structure for insertion into a heart valve of a subject, comprising:
a scaffold panel having a first free end and a second free end when the scaffold panel is in a flattened configuration,
wherein the scaffold panel in the flattened configuration further comprises a first axis spanning from the first free end to the second free end,
wherein the first and second free ends are configured to be coupled to each other to form into a ring when deployed,
wherein the second free end comprises a cantilevered guiding slot,
wherein the first free end comprises a hook that slidably couples to the cantilevered guiding slot in a first direction perpendicular to the first axis, and
wherein sliding the first free end to a terminal position in the first direction deflects the cantilevered guiding slot to retain the hook at the terminal position.

16. The support structure of claim 15, wherein the first free end and the second free end are parallel to each other and perpendicular to the first axis.

17. The support structure of claim 16, wherein the second free end comprises one or more non-return features that restrict further sliding after the first free end and the second free end are slidably coupled.

18. The support structure of claim 17, wherein the one or more non-return features activate after sliding the first free end to the terminal position and rotating the first free und until the hook enters the cantilevered guiding slot.

19. The support structure of claim 17, wherein the second free end comprises one or more of one or more recoil springs or one or more latches.

20. (canceled)

21. The support structure of claim 15, wherein one or more of the first free end or the second free end are not perpendicular to the first axis.

22. (canceled)

23. The support structure of claim 15, wherein the scaffold panel is configured to be loaded into a delivery sheath of a delivery catheter prior to sliding the first free end to a terminal position.

24. The support structure of claim 23, wherein the first free end of the scaffold panel is configured to be rotated counterclockwise prior to loading the scaffold panel into the delivery sheath.

25. The support structure of claim 24, wherein the first free end of the scaffold panel is configured to be rotated clockwise after sliding the first free end to a terminal position in the first direction.

26. The support structure of claim 15, wherein the scaffold panel is configured to be coupled to a valve structure, wherein the scaffold panel is configured to transition the valve structure from a flattened configuration to a ring configuration, wherein the valve structure comprises:
(a) one or more support bodies configured to form a central channel when the valve structure is in the ring configuration;
(b) one or more central leaflet cusps; and
(c) one or more connecting skirts;
wherein the one or more central leaflet cusps are inserted into an arch of the one or more valve support bodies, wherein the one or more connecting skirts are attached to the one or more valve support bodies.

27.-35. (canceled)

36. The support structure of claim 15, further comprising:
a circumferential supporting portion spanning from the first free end to the second free end; and
a plurality of gap filling arms extending from the circumferential supporting portion, wherein at least a terminal portion of the plurality of gap filling arms is parallel and offset from the circumferential supporting portion.

37. The support structure of claim 36, wherein the plurality of gap filling arms defines a cavity around the circumferential supporting portion.

38. The support structure of claim 37, wherein the cavity has a round cross-section or a D-shaped cross-section.

39. (canceled)

40. The support structure of claim 36, wherein the plurality of gap filling arms fills a void between the circumferential supporting portion and an anatomy of the heart valve.

41. The support structure of claim 36, wherein the plurality of gap filling arms creates a paravalvular leak sealing volume.

42. The support structure of claim 36, wherein the plurality of gap filling arms dampens the force of the heart valve on the intraluminal support structure to allow the intraluminal support structure to maintain a cylindrical valve orifice.

43. (canceled)

44. The support structure of claim 36, wherein the support structure is non-deformable.

45. The support structure of claim 36, wherein the support structure is deformable.

46. The support structure of claim 45, wherein the intraluminal support structure is larger than the heart valve in an anterior-posterior direction, wherein the intraluminal support structure exerts force on the heart valve, wherein the gap filling arms are pushed outward.

47. The support structure of claim 36, wherein the gap filling arms have an open toroidal braided structure.

48. (canceled)
